(19) United States
(12) Patent Application Publication
(10) Pub. No.: US 2014/0186386 A1
(43) Pub. Date: Jul. 3, 2014

(54) METHODS FOR PREVENTING AND TREATING STAPHYLOCOCCUS AUREUS COLONIZATION, INFECTION, AND DISEASE

(71) Applicants: Jeffrey Weiser, Merion, PA (US); Rebeccah S. Lijek, Cambridge, MA (US)

(72) Inventors: Jeffrey Weiser, Merion, PA (US); Rebeccah S. Lijek, Cambridge, MA (US)

(21) Appl. No.: 14/122,605

(22) PCT Filed: Jun. 5, 2012
(86) PCT No.: PCT/US12/40865

§ 371 Date: Mar. 3, 2014

Related U.S. Application Data
(60) Provisional application No. 61/493,900, filed on Jun. 6, 2011.

(51) Int. Cl. A61K 39/085; A61K 9/00

(52) U.S. Cl. CPC A61K 39/085; A61K 9/007
USPC 424/190.1

(57) ABSTRACT

The invention provided herein relates to a method of preventing a Staphylococcus aureus colonization and/or infection by administering a Streptococcus pneumoniae strain, antigen thereof, or homologous staphylococcal antigen. The invention further relates to a method of treating a disease associated with a Staphylococcus aureus colonization and/or infection by administering a Streptococcus pneumoniae strain, antigen thereof, or homologous staphylococcal antigen.

### GOVERNMENT INTEREST

The work described herein was supported, in part, by a grant from National Institute of Health (Grant Number T32 AI055400). The United States government may have certain rights in this application.

### FIELD OF THE INVENTION

The invention relates to methods for treating diseases associated with Staphylococcus aureus colonization and/or infection. Specifically, the invention relates to preventing Staphylococcus aureus colonization and/or infection by administering a vaccine composition comprising a recombinant SP_{—}1119 protein, or a recombinant P5CDH protein or a combination thereof.

### BACKGROUND OF THE INVENTION

Staphylococcus causes several diseases by various pathogenic mechanisms. The most frequent and serious of these diseases are bacteremia and its complications in hospitalized patients. In particular, Staphylococcus can cause wound infections and infections associated with catheters and prosthetic devices. Serious infections associated with Staphylococcus bacteremia include osteomyelitis, invasive endocarditis and septicemia. The problem is compounded by multiple antibiotic resistance in hospital strains, which severely limits the choice of therapy. In the majority of cases the causative organism is a strain of S. aureus, S. epidermidis, S. haemolyticus or S. hominis, or a combination of these. The problem with Staphylococcus is compounded by multiple antibiotic resistance in hospital strains, which severely limits the choice of therapy.

There are numerous methicillin-susceptible (MSSA) and methicillin-resistant (MRSA) strains. The Number of people infected with MRSA each year is 880,000 and the 2007 number of MRSA infection deaths per year is 20,000 to 40,000. The emergence of drug resistance has made many of the available antimicrobial agents ineffective. Therefore, alternative methods for the prevention and treatment of bacterial infections in general and S. aureus infections in particular are needed.

### SUMMARY OF THE INVENTION

The invention provided herein relates to a vaccine composition comprising a recombinant protein having the amino acid sequence set forth in SEQ ID NO.: 1 or 2, wherein said composition is capable of preventing Staphylococcus aureus (S. aureus) colonization and/or infection in a subject.

The invention provided herein also relates to a method of preventing a Staphylococcus aureus (S. aureus) colonization and/or infection in a subject, the method comprising the step of administering a therapeutically effective amount of a vaccine composition that comprises a recombinant protein having the amino acid sequence set forth in SEQ ID NO.: 1 or 2, thereby preventing colonization and/or infection in the subject.

The invention provided herein further relates to a method of treating a disease associated with Staphylococcus aureus (S. aureus) colonization and/or infection in a subject, in another embodiment, the method comprising the step of administering a therapeutically effective amount of a vaccine composition that comprises a recombinant protein having the amino acid sequence set forth in SEQ ID NO.: 1 or 2, thereby treating said disease in the subject.

The invention provided herein further relates to a method of eliciting an anti-S. aureus immune response in a subject, said method comprising the step of administering to said subject a therapeutically effective amount of a vaccine composition comprising a recombinant protein having the amino acid sequence set forth in SEQ ID NO.: 1 or 2, thereby eliciting said anti-S. aureus immune response in the subject.

The invention provided herein further relates a method of preventing a Staphylococcus aureus (S. aureus) colonization and/or infection in a subject, wherein the method comprises the step of administering a therapeutically effective amount of a first vaccine composition that comprises a recombinant protein having the amino acid sequence set forth in SEQ ID NO.: 1 and a second vaccine composition that comprises a recombinant protein having the amino acid sequence set forth in SEQ ID NO.: 2, thereby preventing said colonization and/or infection in the subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings.

FIG. 1. Shows that pneumococcal colonization induces antibodies that cross-react with S. aureus surface proteins. A) Shows surface-bound IgG before (orange) and after (red) pneumococcal colonization as measured by flow cytrometry using FITC-conjugated anti-mouse IgG. Gray shaded: no primary sera control. B) Quantification of (A). C) and D) Show a candidate 54 kD S. aureus surface protein recognized using western blots (C) and 2-D westerblot (D) of S. aureus whole cell lysates probed with sera from mice pre- and post-colonization with S. pneumoriae (Sp) or sham (PBS).

FIG. 2. Shows that S. Aureus antigens are surface-exposed; S. Pneumoniae antigens are surface-associated but masked by capsule. A) Live S. aureus 8325-4 Δspa cells were incubated with sera from rabbits before (gray solid) and after (black line) immunization with either heat-killed whole cell S. aureus (αHKSa, positive control) or purified recombinant S. aureus antigens (P5CDH or DLDH). Surface-bound IgG was measured by flow cytometry using FITC-conjugated anti-rabbit IgG and increase in surface binding is demonstrated by the increase along the X-axis. B) Live S. pneumoniae TIGR4 cells were incubated with sera from rabbits before (gray solid) and after (black line) immunization with either heat-killed whole cell S. pneumoniae TIGR4 (αHKSp, positive control) or purified recombinant S. pneumoniae antigens (SP_{—}1119 or SP_{—}1161). Surface-bound IgG was measured by flow cytometry using FITC-conjugated anti-rabbit IgG. C) Same as (B) using live unencapsulated S. pneumoniae TIGR4 Δcps cells.

FIG. 3. Shows that antibodies raised against pneumococcal protein SP_{—}1119, but not SP_{—}1161, bind to the surface of S. aureus. A) Live S. aureus 8325-4 Δspa cells were incubated with sera from rabbits before (gray solid) and after (black line) immunization with purified recombinant S. pneumoniae antigens (SP_{—}1119 or SP_{—}1161). Surface-bound IgG was measured by flow cytometry using FITC-conjugated anti-rabbit IgG. B) Quantification of specific antibody binding to the surface of live S. aureus 8325-4 Δspa. C) Live S. pneumoniae TIGR4Δcps cells were incubated with sera from rabbits before (gray solid) and after (black line) immunization with purified recombinant S. aureus antigens (P5CDH or DLDH).

FIG. 4. Shows that pneumococcal colonization elicits antibodies that cross-react with S. aureus protein P5CDH. Western blot of purified recombinant proteins SP_{—}1119 and SP_{—}1161 from S. pneumoniae and P5CDH and DLDH from S. aureus probed with sera from mice pre- and post-colonization with S. pneumoniae TIGR4 (Sp) or sham (PBS). Blots from two S. pneumoniae-colonized animals are pictured in order to display the observed inter-animal variability in antibody responses.

FIG. 5. Shows that S. aureus antigens DLDH and P5CDH are broadly conserved. Western blot of S. aureus whole cell lysates representing a diversity of important clinical isolates as well as commonly used lab strains. Lysates were probed with sera from pre- and post-immunization with purified recombinant proteins DLDH and P5CDH. MSSA, methicillin-susceptible S. aureus; MRSA, methicillin-resistant S. aureus.

FIG. 6 shows a role for the host immune system in mediating S. aureus colonization. The antibody response to S. pneumoniae colonization cross-reacts with specific S. aureus antigens, leading to protection in vivo.

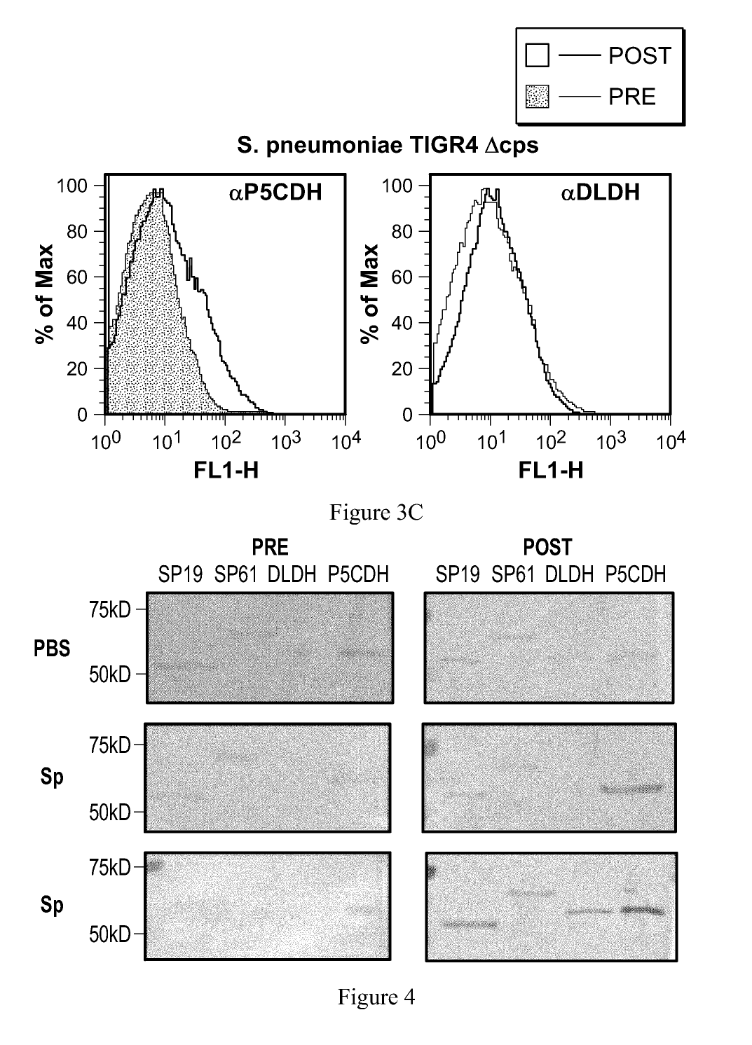

FIG. 7 shows generation of specific tools, according to one embodiment of the invention.

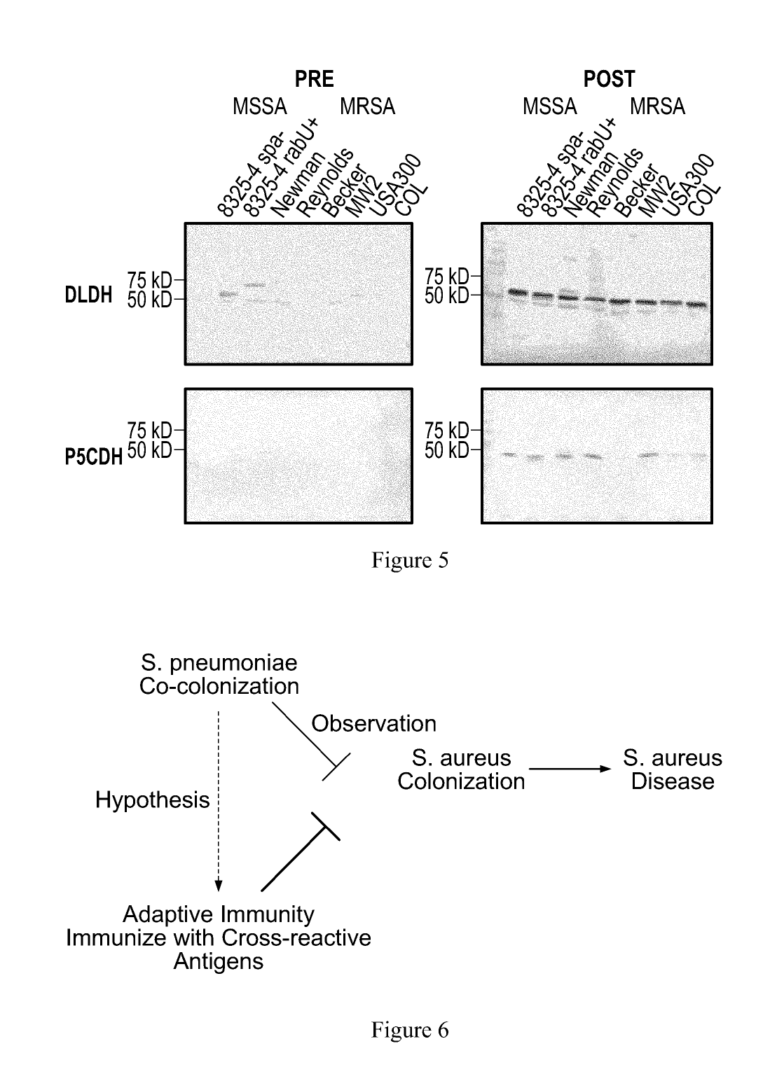

FIG. 8 shows that S. aureus antigens are surface-exposed, according to one embodiment of the invention.

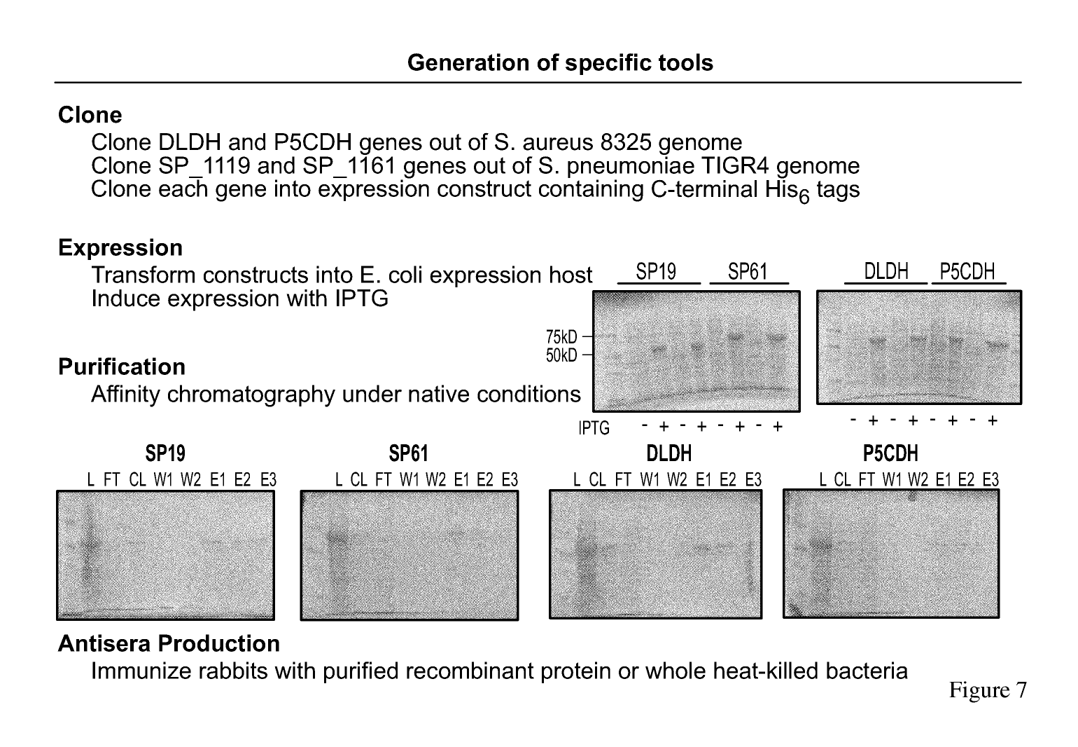

FIG. 9 shows confirming specificity of cross-reactivity. In vitro approach was used to determine whether specific antisera cross-react with recombinant proteins. His tags were removed by thrombin cleavage. Western blots, ELISA of recombinant proteins with/without His tags using rabbit antisera was raised against specific antigens. Our results shoes that there were no cross-reactive bands to recombinant proteins without His tags. In vivo approach was used to determine whether bacterial mutants lacking SP19, P5CDH lose cross-reactivity. Deletion mutations were made in specific antigens. Double deletion mutant in SP_{—}1119 was constructed. It was observed that the loss of SP_{—}1119 results in loss of cross-reactive binding by antisera to P5CDH.

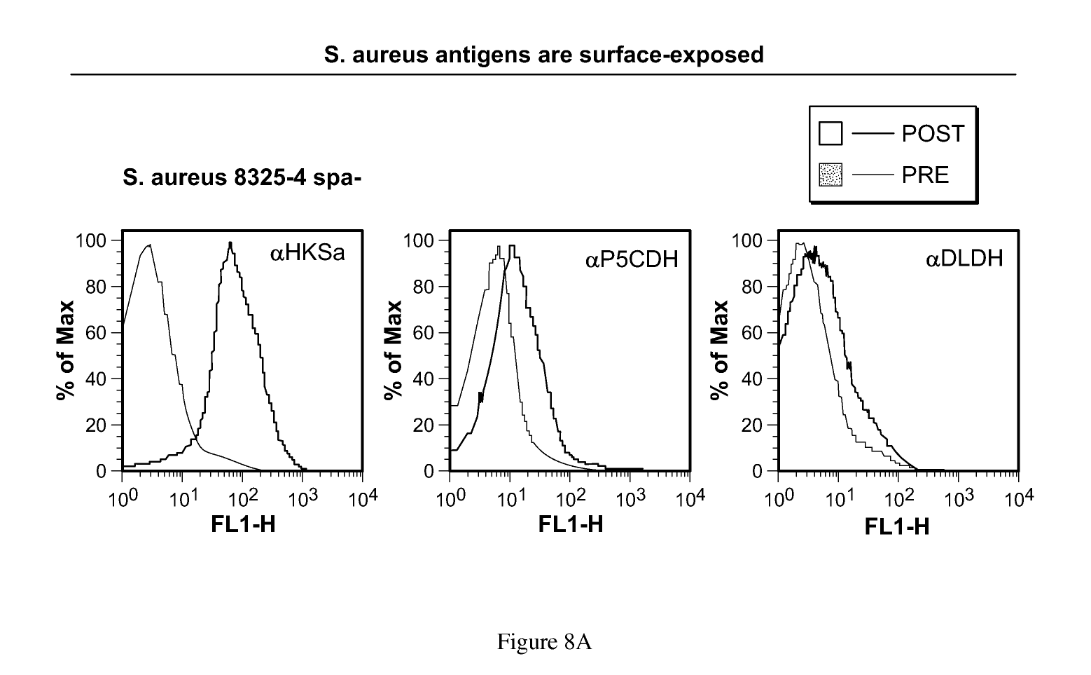

FIG. 10 shows that pneumococcal colonization elicits antibodies that recognize SP_{—}1119 and P5CDH.

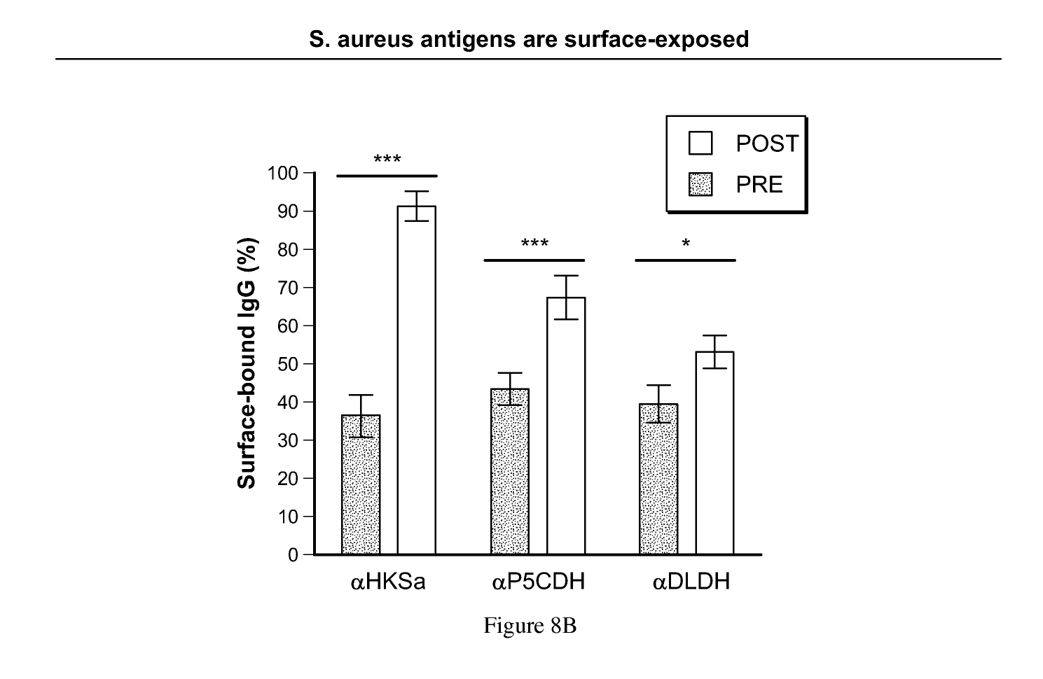

FIG. 11 illustrates an experiment to determine whether children colonized with S. pneumoniae mount antibody titers to candidate antigens.

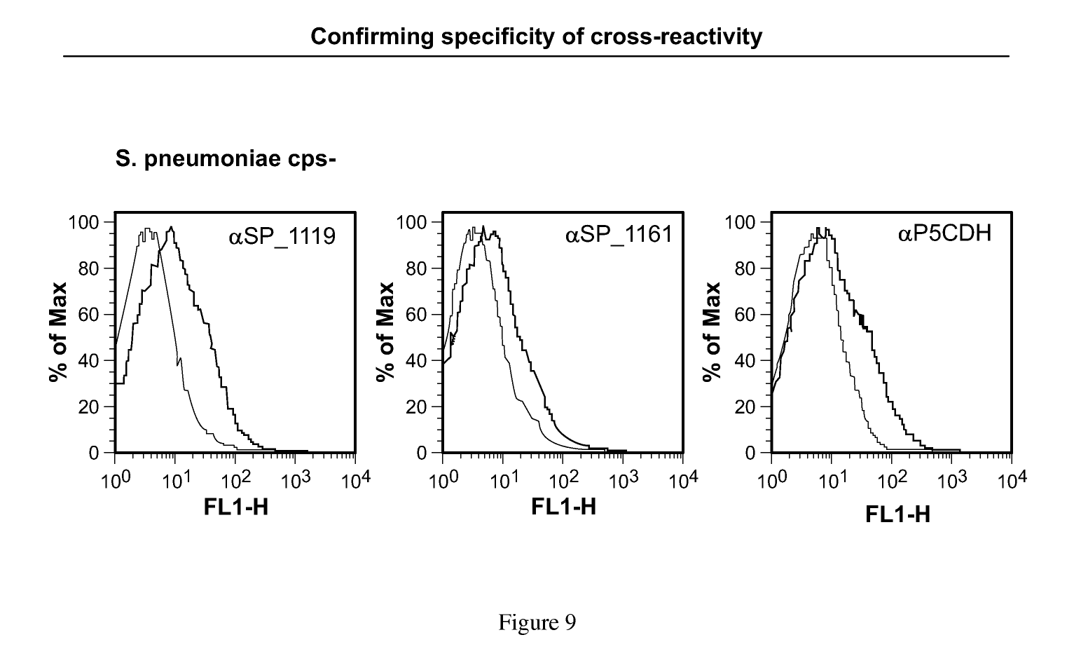

FIG. 12 shows that SP_{—}1119 and SP_{—}1161 are immunogenic in human children. Pneumococcal carriage correlated with higher titers to SP19 (trend at 12 mo, p=0.0002 at 24 months) and SP61 (p=0.019 at 12 mo, trend at 24 mo).

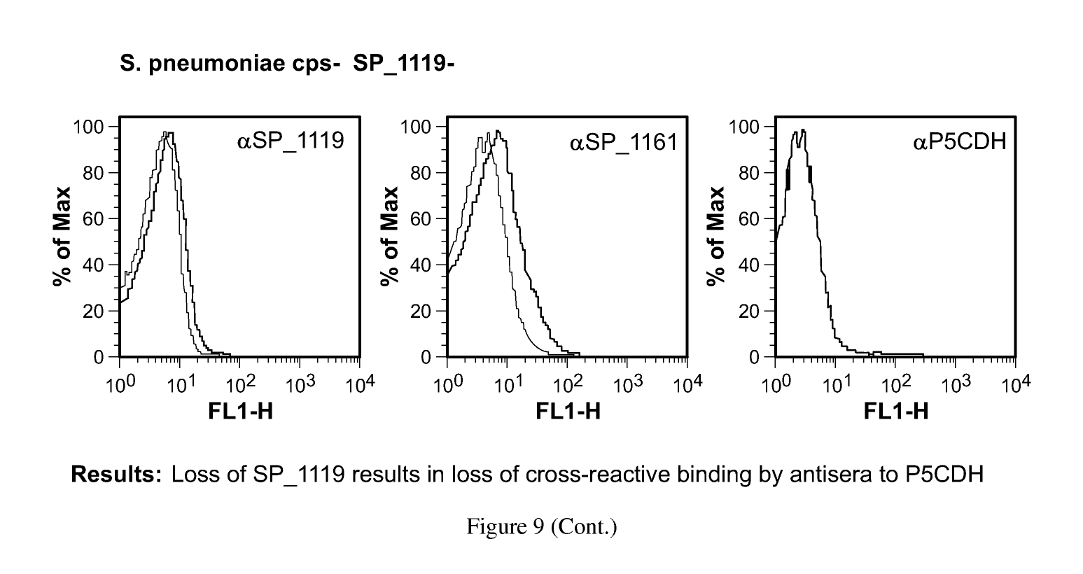

FIG. 13 shows a working model, according one embodiment of the invention.

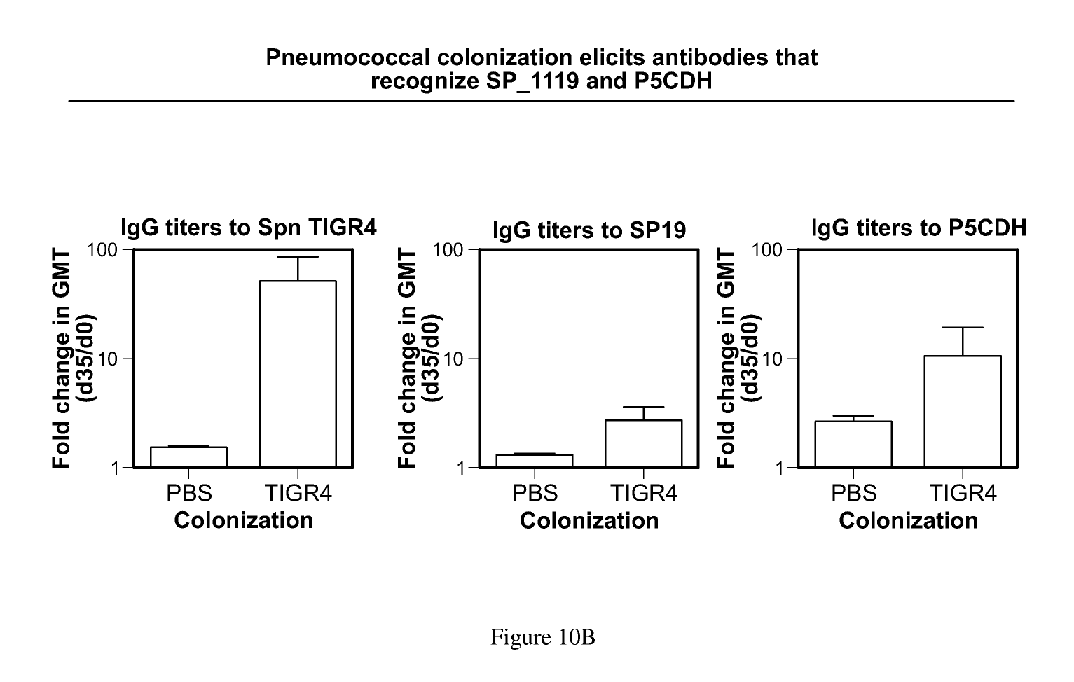

FIG. 14 shows a working model, according to another embodiment of the invention.

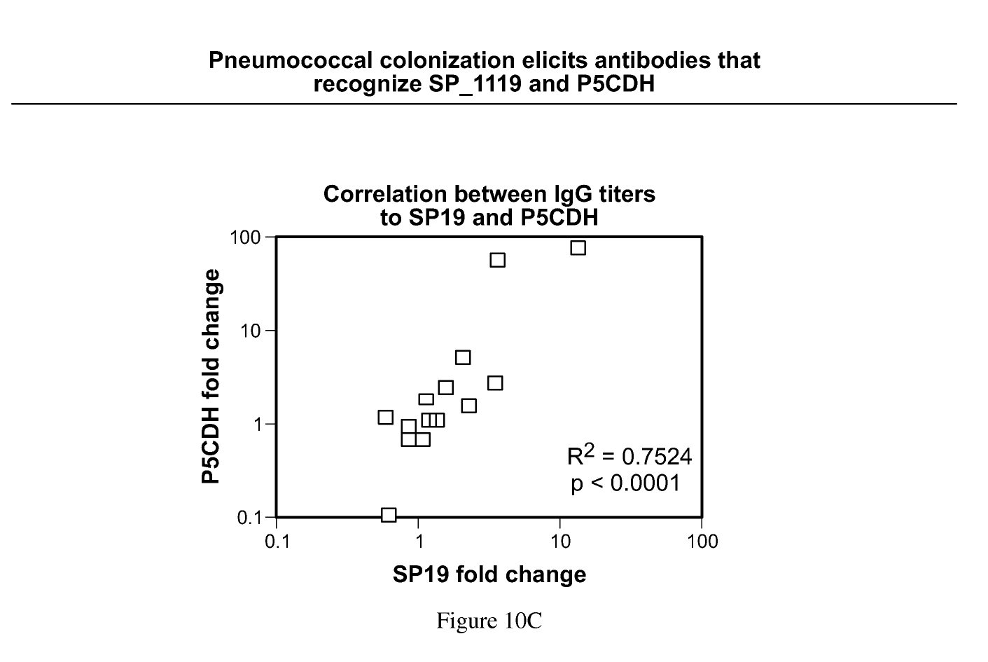

FIG. 15 shows protection against S. aureus. Opsonophagocytic killing of S. aureus by human neutrophils ex vivo had no effect of killing with: any rabbit antisera, adherent human neutrophils, or HL-60 neutrophil-like cell line. Antibody-mediated complement deposition on S. aureus surface had no effect of any other rabbit antisera or bacterial uptake once opsonized.

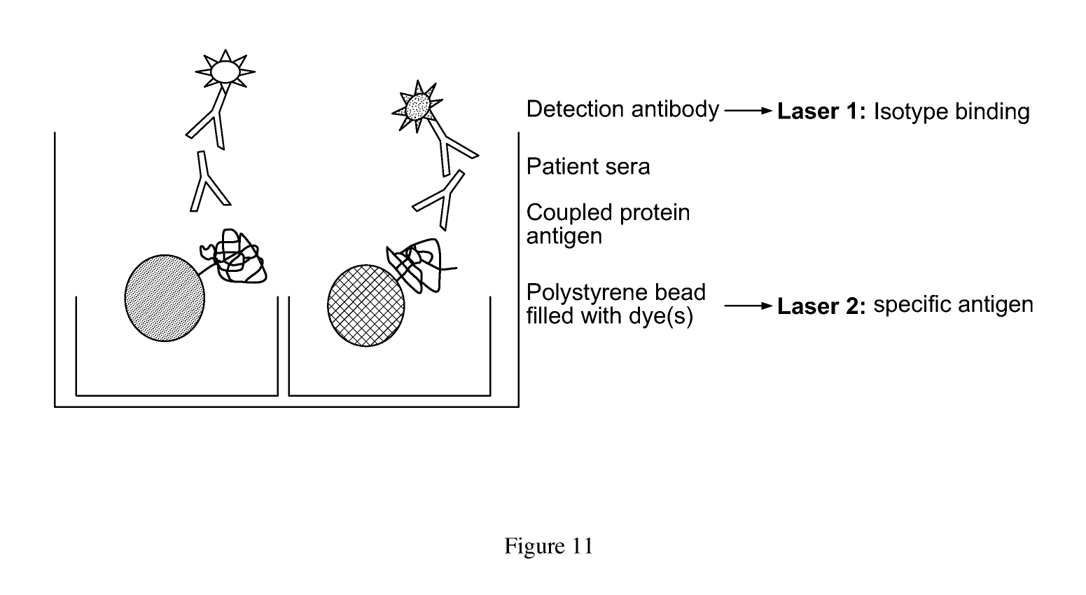

FIG. 16 shows protection against S. aureus in vitro. Antibody to P5CDH and SP_{—}1119, but not DLDH and SP_{—}1161 inhibits growth of S. aureus.

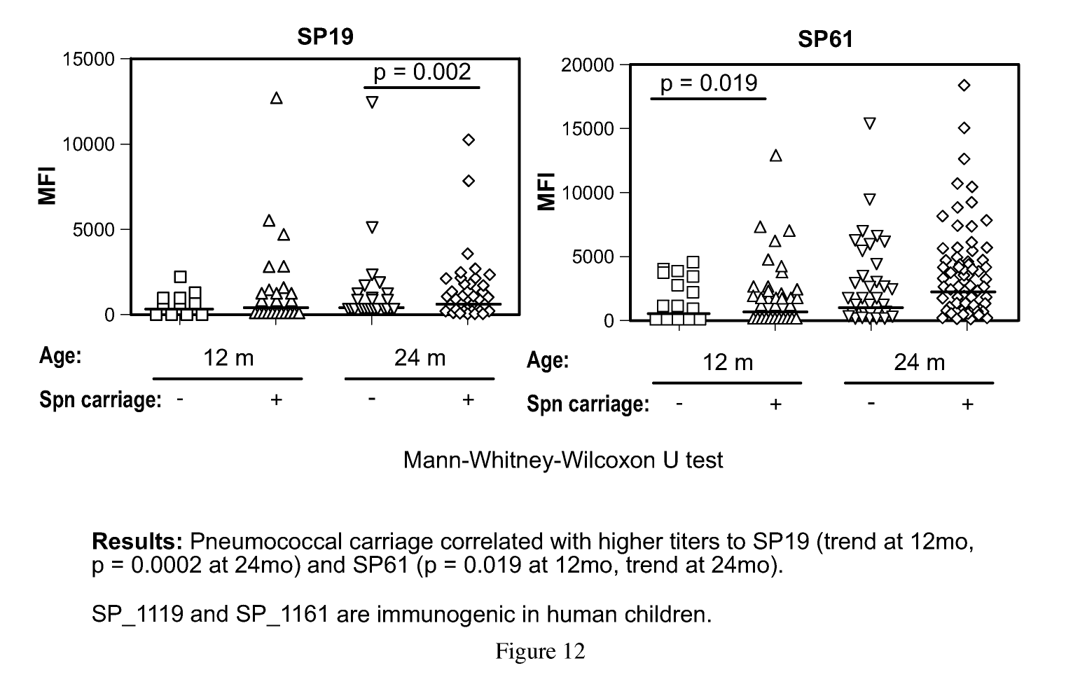

FIG. 17 shows murine model of S. aureus bacteremia, according to one embodiment of the invention.

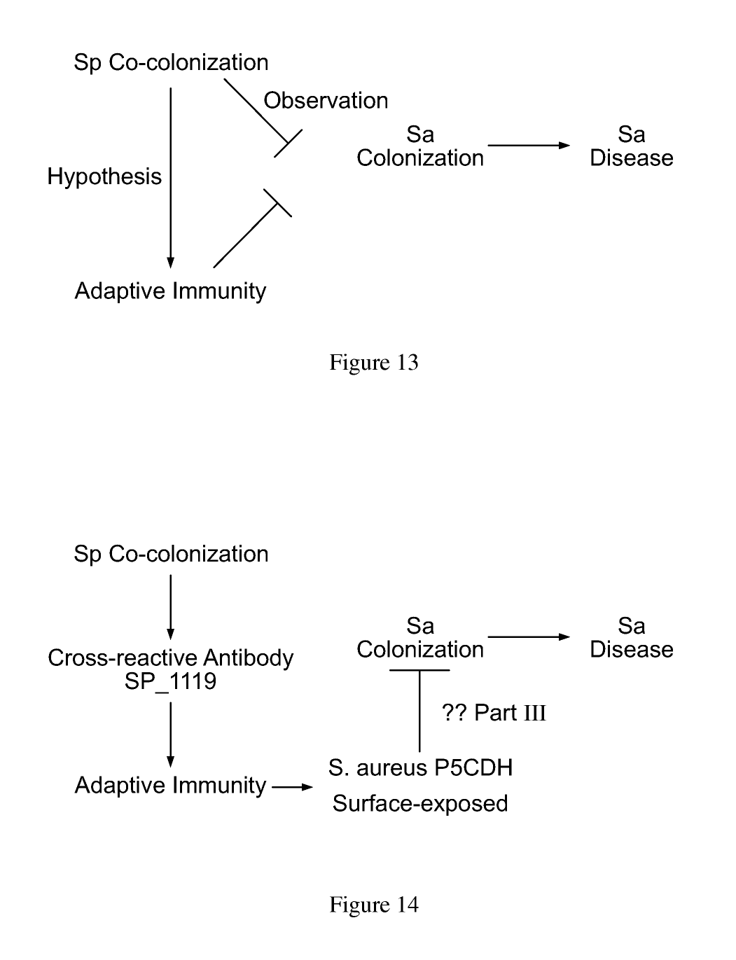

FIG. 18 shows survival of S. aureus Newman bacteremia following passive immunization.

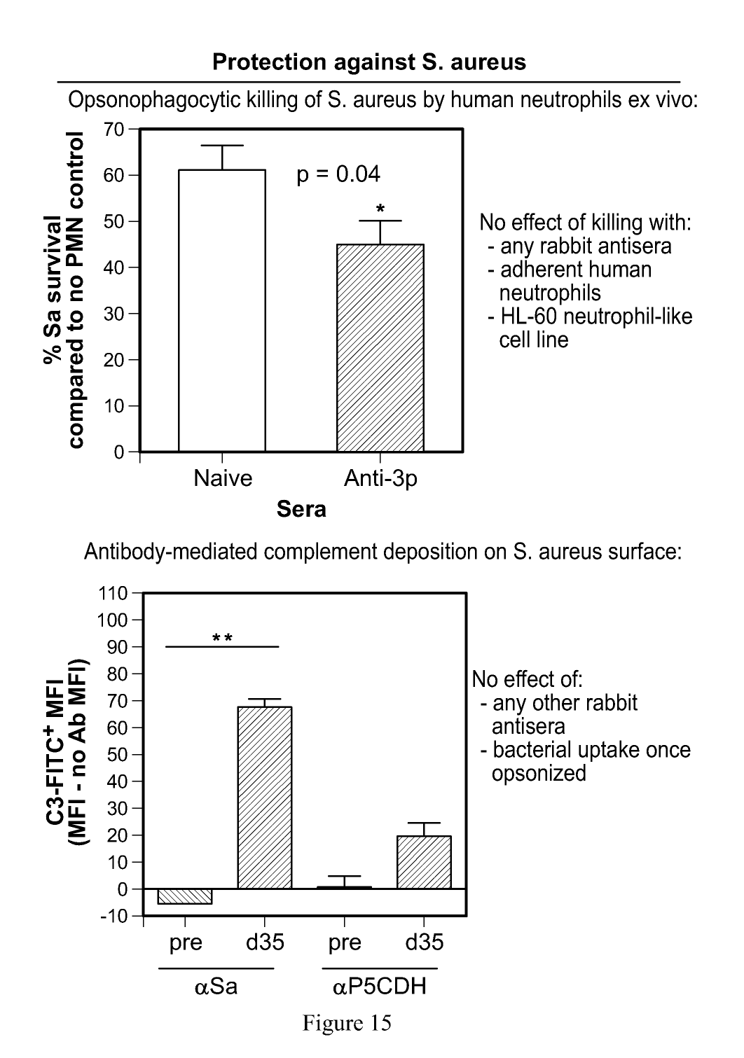

FIG. 19 shows bacteremic burden following passive immunization. The results show significant reduction in bacterial burden in blood after αP5CDH passive immunization.

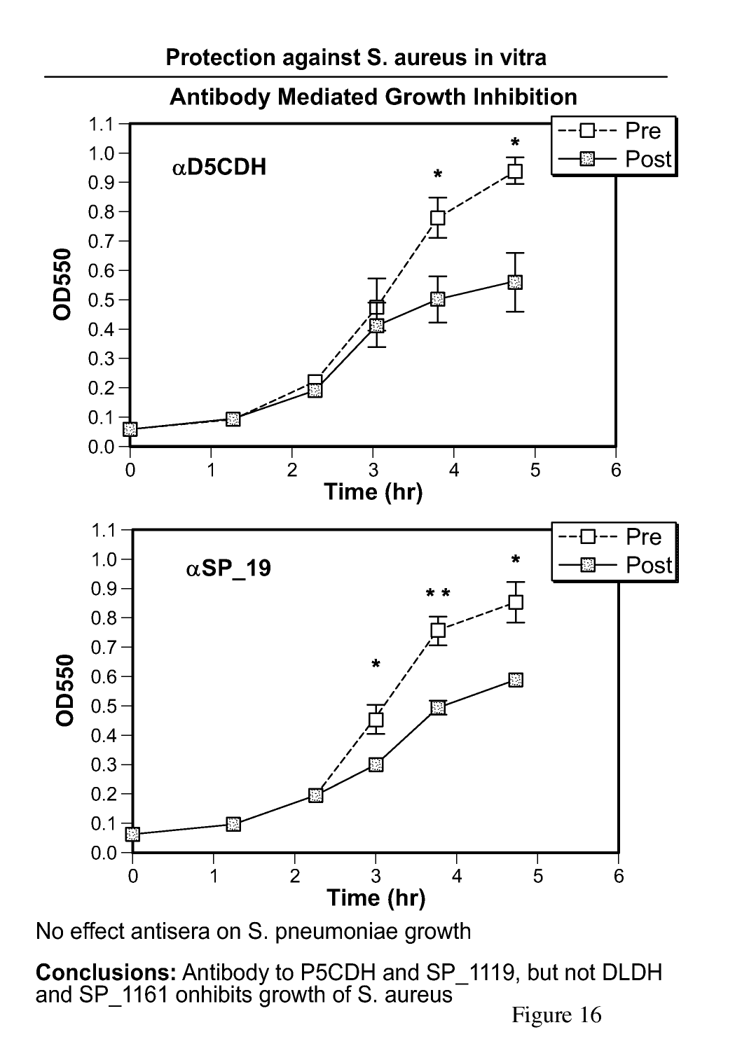

FIG. 20 shows S. aureus Newman colonization in CD1 mice at day 2. Our results showed poor colonization. Our results showed no effect on inoculation dosage and antibiotic selection in vivo. Additionally, our results showed no difference between lavage v. nasal tissue excision.

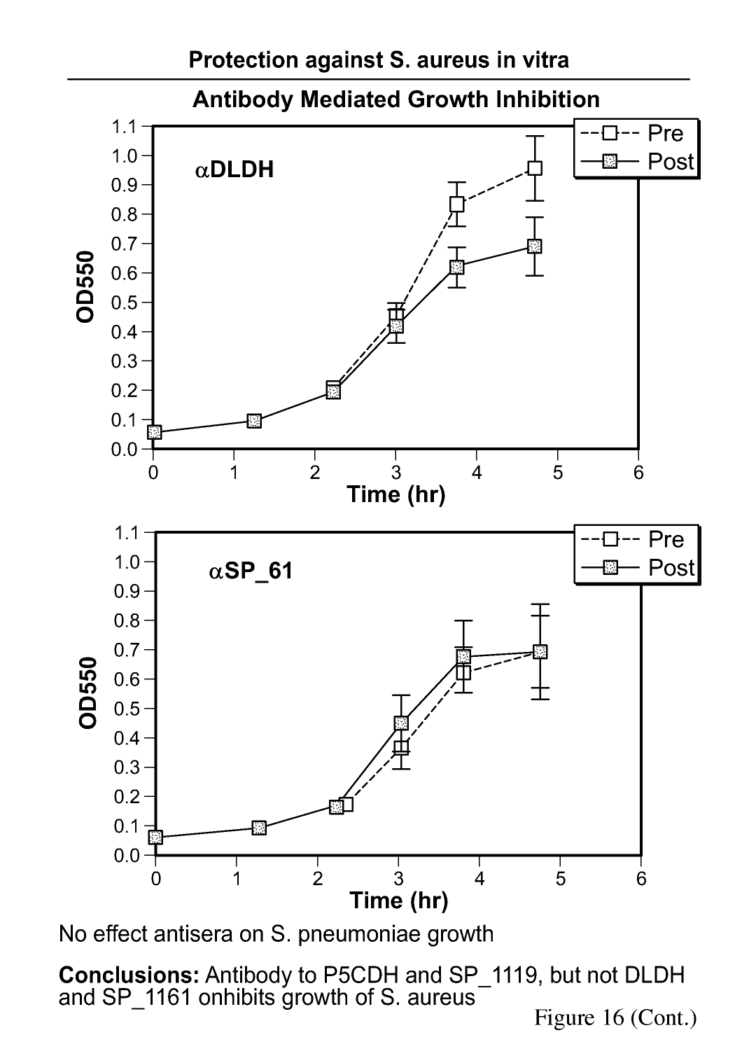

FIG. 21 shows that S. aureus 502A expresses DLDH and P5CDH during log- and stationary phase.

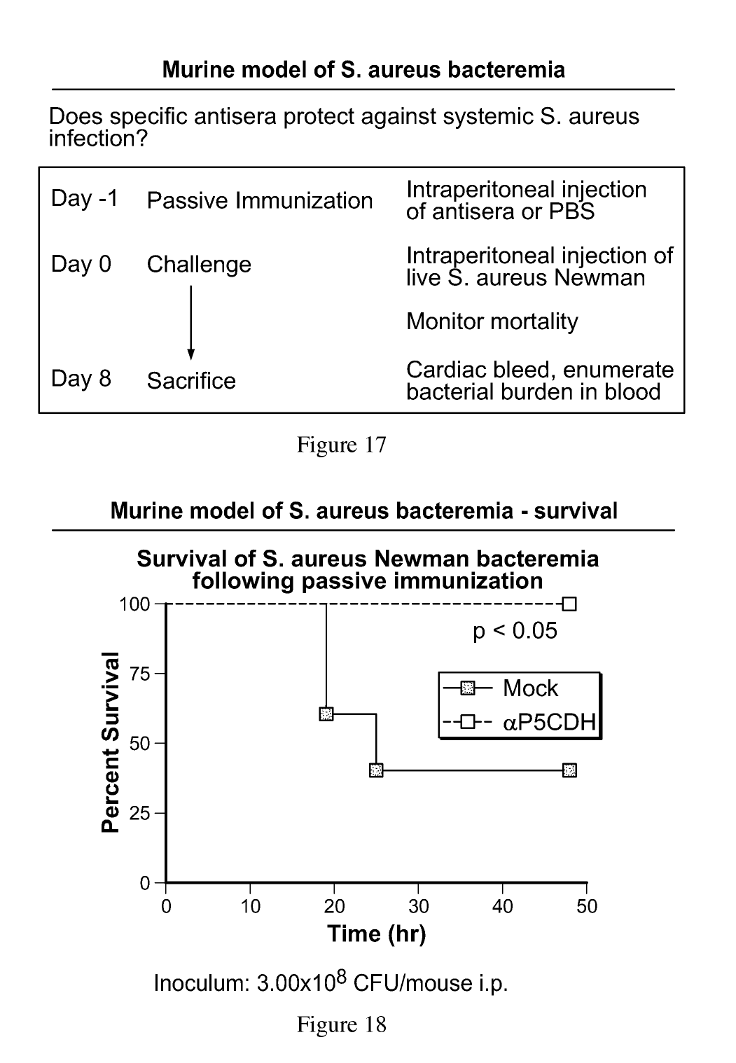

FIG. 22 shows nasal colonization of S. aureus 502A. Similar colonization levels in outbred CD-1, antibody deficient μMT. It was observed that, at higher levels, 502A colonizes more consistently than Newman. It was also observed that all animals carry 502A at day 1, reduced levels from day 2-3.

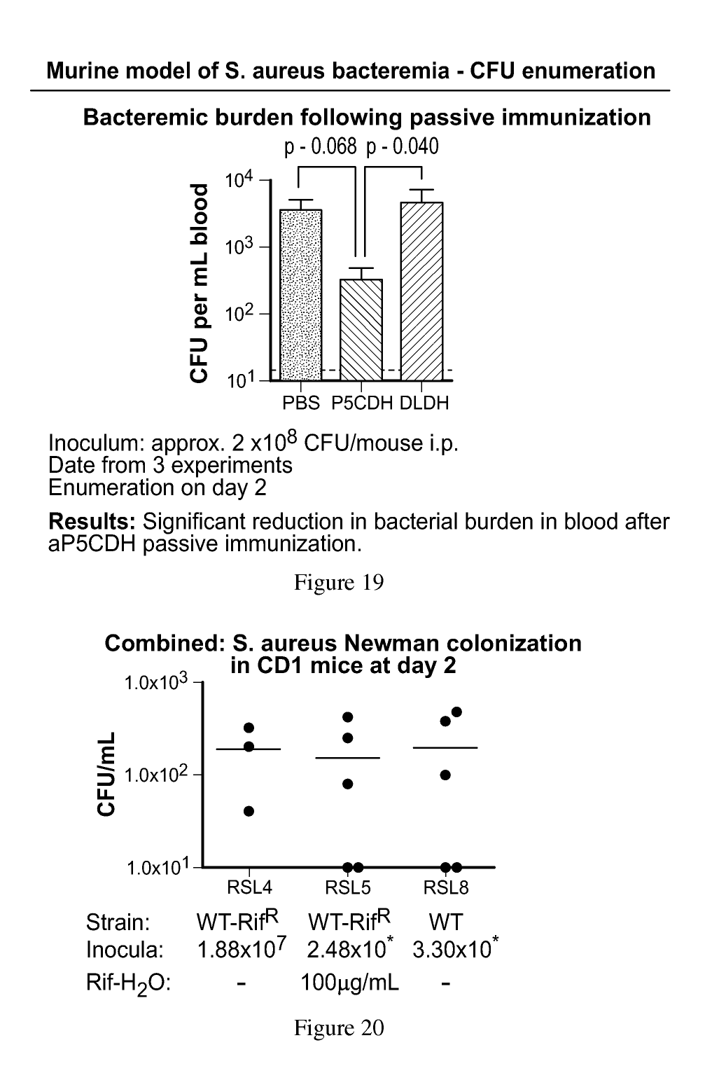

FIG. 23 shows colonization of S. aureus 502A. Colonization with S. pneumoniae against subsequent S. aureus carriage in an antibody-dependent manner (pending more μMTs).

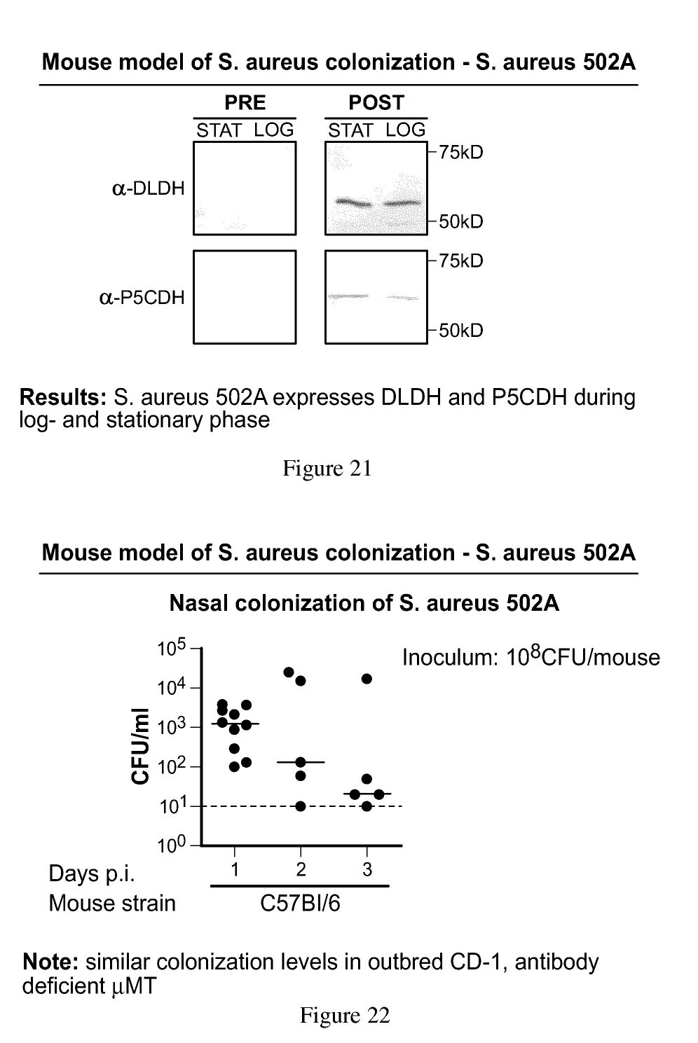

FIG. 24 shows a working model, according to one embodiment of the invention.

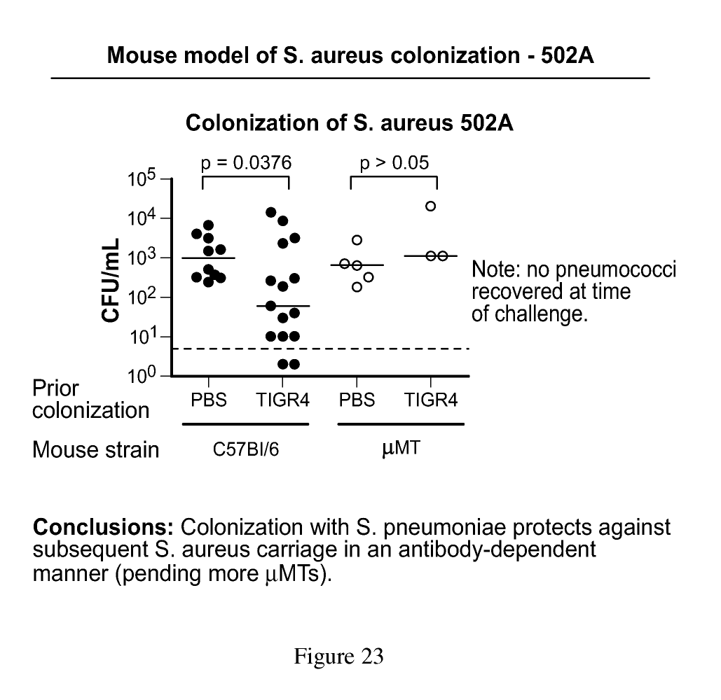

FIG. 25 shows a working model, according to another embodiment of the invention.

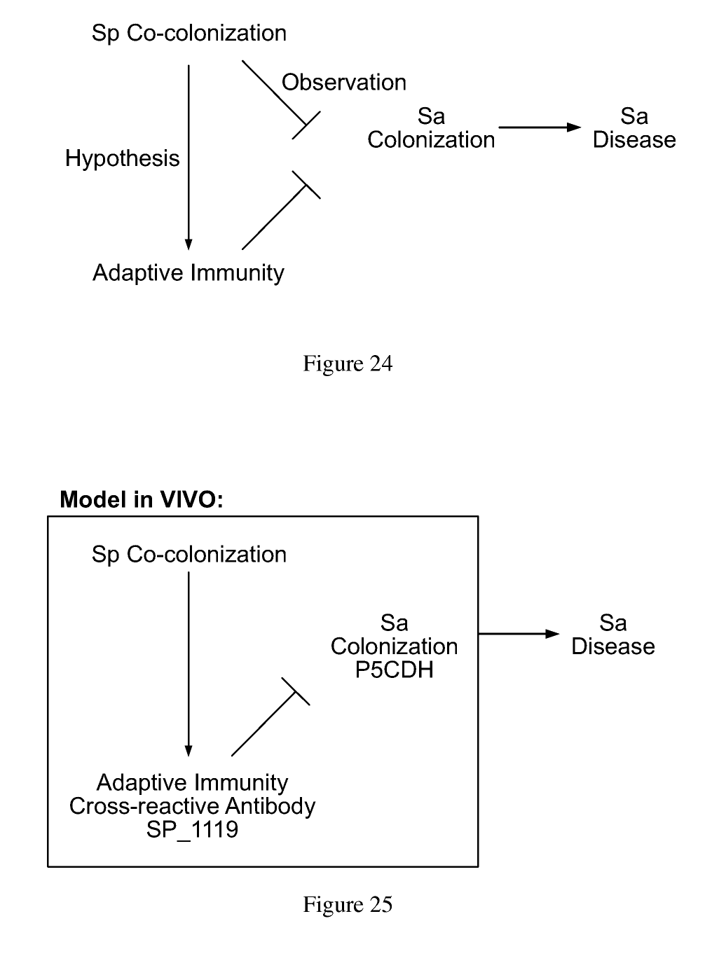

FIG. 26 shows that immunization with SP_{—}1119 significantly reduces S. aureus 502A nasal carriage at day 1 post challenge compared to adjuvant-only controls. C57B1/6 mice were immunized intranasally once a week for three weeks with purified antigens plus cholera toxin as adjuvant. Two weeks following the last immunization, mice were challenged intranasally with S. aureus 502A. At day 1 post-challenge, nasal lavages were taken to enumerate CFU of S. aureus 502A.

### DETAILED DESCRIPTION OF THE INVENTION

The invention provided herein relates to methods of using the vaccine compositions disclosed herein in a prophylactically and therapeutically effective manner to prevent and/or treat disease. Thus, the present invention is also directed to a method of preventing or attenuating a colonization and/or infection caused by a member of the genus Staphylococcus in a subject (e.g., human), comprising administering to the subject a therapeutically effective amount of a vaccine composition as disclosed herein according to the present invention.

In one embodiment, provided herein is a vaccine composition comprising a recombinant protein having the amino acid sequence set forth in SEQ ID NO.: 1 or 2, wherein said composition is capable of preventing Staphylococcus aureus (S. aureus) colonization and/or infection in a subject.

In one embodiment, SEQ ID NO: 1 represents the sequence of SP_{—}1119 (Genbank ID: NP_{—}345590.1) as follows:

mtryqnlvng kwksseqeit iyspinqeel gtvpamtqte adeamqaara alpawralsa veraaylhkt
aailerdkee igtilakeva kgikaaigev vrtadlirya aeeglritgq amegggfeat sknklavvrr
epvgivlaia pfnypvnlsa skiapaliag nvvmfkpptq gsisglllak afeeagipag vfntitgrgs
eigdyiiehk evnfinftgs tpigerigrl agmrpimlel ggkdaalvle dadlehaakq ivagafsysg
qrctaikrvi vlesvadkla tllqeevskl tvgdpfdnad itpvidnasa dfiwglieda qekeaqaltp
ikregnllwp vlfdqvtkdm kvaweepfgp vlpiirvasv eeaiafanes efglqssvft ndfkkafeia
eklevgtvhi nnktqrgpdn fpflgvkgsg agvqgikysi eamtnvksiv fdvk

In one embodiment, SEQ ID NO: 2 represents the sequence of P5CDH (Genbank ID: YP_{—}501325.1) as follows:

mvvefknepg ydfsvqenvd mfkkalkdve kelgqdiplv ingekifkdd kiksinpadt sqvianaska
tkqdvedafk aaneaykswk twsandrael mlrvsaiirr rkaeiaaimv yeagkpwdea vgdaaegidf
ieyyarsmmd laqgkpvldr egehnkyfyk sigtgvtipp wnfpfaimag ttlapvvagn tvllkpaedt
pyiayklmei leeaglpkgv vnfvpgdpke igdylvdhkd thfvtftgsr atgtriyers avvqegqnfl
krviaemggk daivvdenid tdmaaeaivt safgfsgqkc sacsraivhk dvydevleks ikltkeltlg
ntvdntymgp vinkkqfdki knyieigkee gkleqgggtd dskgyfvept iisglkskdr imqeeifgpv
vgfvkvndfd eaievandtd ygltgavitn nrehwikavn efdvgnlyln rgctsavvgy hpfggfkmsg
tdaktgspdy llhfleqkvv semf

In another embodiment, provided herein is a method of preventing a Staphylococcus aureus (S. aureus) colonization and/or infection in a subject, the method comprising the step of administering a therapeutically effective amount of a vaccine composition that comprises a recombinant protein having the amino acid sequence set forth in SEQ ID NO.: 1 or 2, thereby preventing colonization and/or infection in the subject.

In one embodiment, provided herein is a method of treating a disease associated with Staphylococcus aureus (S. aureus) colonization and/or infection in a subject, in another embodiment, the method comprising the step of administering a therapeutically effective amount of a vaccine composition that comprises a recombinant protein having the amino acid sequence set forth in SEQ ID NO.: 1 or 2, thereby treating said disease in said subject.

In another embodiment, provided herein is a method of eliciting an anti-S. aureus immune response in a subject, said method comprising the step of administering to said subject a therapeutically effective amount of a vaccine composition comprising a recombinant protein having the amino acid sequence set forth in SEQ ID NO.: 1 or 2, thereby eliciting said anti-S. aureus immune response in said subject.

In one embodiment provided herein is a method of preventing a Staphylococcus aureus (S. aureus) colonization and/or infection in a subject, wherein the method comprises the step of administering a therapeutically effective amount of a first vaccine composition that comprises a recombinant protein having the amino acid sequence set forth in SEQ ID NO.: 1 and a second vaccine composition that comprises a recombinant protein having the amino acid sequence set forth in SEQ ID NO.: 2, thereby preventing said colonization and/or infection in said subject.

The invention provided herein relates in another embodiment to a method of preventing a Staphylococcus aureus (S. aureus) colonization and/or infection in a subject, the method comprising the step of administering a therapeutically effective amount of a vaccine composition comprising a recombinant SP19 protein, or a recombinant P5CDH protein, or a combination thereof; wherein administering said vaccine to said subject enables an initial humoral response against a S. aureaus colonization and/or infection in said subject.

The invention also relates to a method of treating a Staphylococcus aureus (S. aureus) colonization and/or infection in a subject, the method comprising the step of administering a therapeutically effective amount of a vaccine composition comprising a recombinant SP19 protein, or a recombinant P5CDH protein, or a combination thereof; wherein administering said vaccine to said subject enables an initial humoral response against a S. aureaus colonization and/or infection in said subject.

The invention further relates to a method of eliciting an anti-S. aureus immune response in a subject, the method comprising the step of administering a therapeutically effective amount of a vaccine composition comprising a recombinant SP19 protein, or a recombinant P5CDH protein, or a combination thereof; wherein administering said vaccine to said subject enables an initial humoral response against a S. aureaus colonization and/or infection in said subject.

In another embodiment, the invention provides a method of vaccinating a subject for the prevention or treatment of an S. aureus colonization and/or infection according to the methods above. In another embodiment, administration of the vaccine composition provided herein to a host or subject elicits a therapeutic response against an S. aureus colonization and/or infection in the host or subject.

In one embodiment, the term “infection,” as used herein, may refer to colonization, infection, and/or asymtomatic carriage.

In one embodiment, the therapeutic response is a humoral response is a humoral response therapeutic against an S. aureus colonization and/or infection. In another embodiment the immune response is an adaptive response against an S. aureus colonization and/or infection. In one embodiment, the term “Antigenic fragment” of a protein refers to a portion of such a protein which is capable of binding an antibody.

In another embodiment, the SP19 and P5CDH recombinant proteins are purified from of S. pneumoniae native antigens SP_{—}1119 and P5CDH, respectively. In another embodiment, the antigen is SP_{—}1161. In another embodiment, the antigen is DLDH. The present invention also encompasses the use of one or more S. pneumoniae cell wall and/or cell membrane proteins and/or immunogenically-active fragments, derivatives or modifications thereof in the preparation of a vaccine for use in the prevention of S. aureaus colonization and/or infection. The S. pneumoniae cell-wall and/or cell-membrane proteins for use in working the present invention may be obtained by directly purifying the proteins from cultures of S. pneumoniae by any of the standard techniques used to prepare and purify cell-surface proteins. Suitable methods are described in many biochemistry text-books, review articles and laboratory guides, including inter alia “Protein Structure: a practical approach” ed. T. E. Creighton, IRL Press, Oxford, UK (1989). In another embodiment, the S. pneumoniae SP_{—}1119 is homologous to 1-pyrroline-5-carboxylate dehydrogenase (P5CDH) of S. aureus. In another embodiment, the S. pneumoniae SP_{—}1161 is homologous to dihydrolipoamide dehydrogenase (DLDH) of S. aureus.

In another embodiment, the humoral response elicited by administering S. pneumoniae to a host or subject is an antibody response against a vaccine composition provided herein. In another embodiment, the humoral response elicited by administering S. pneumoniae to a host or subject is an antibody response against a recombinant protein or a fragment thereof, further provided herein. In another embodiment, the humoral response is a polyclonal antibody response. In another embodiment, an antibody from the humoral response is an immunoglobulin G.

In another embodiment, a vaccine of the present invention further comprises an adjuvant. The adjuvant utilized in methods and compositions of the present invention is, in another embodiment, a cholera toxin. In another embodiment, the adjuvant comprises a a granulocyte/macrophage colony-stimulating factor (GM-CSF) protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art. Each possibility represents a separate embodiment of the present invention.

In one embodiment, the terms “antibody” and “immunoglobulin” are used interchangeably herein. These terms are well understood by those in the field, and refer to a glycosylated (comprising sugar moieties) protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

By “binds specifically” is meant high avidity and/or high affinity binding of an antibody to a specific polypeptide, e.g., epitope of a protein. Antibody binding to its epitope on this specific polypeptide is preferably stronger than binding of the same antibody to any other epitope, particularly those which may be present in molecules in association with, or in the same sample, as the specific polypeptide of interest, e.g., binds more strongly to epitope fragments of a target protein, such as one provided herein, so that by adjusting binding conditions the antibody binds almost exclusively to an epitope site or fragments of a desired protein.

By “detectably labeled antibody” is meant an antibody (or antibody fragment which retains binding specificity), having an attached detectable label. The detectable label is normally attached by chemical conjugation, but where the label is a polypeptide, it could alternatively be attached by genetic engineering techniques. Methods for production of detectably labeled proteins are well known in the art. Detectable labels known in the art include radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin), methods for labeling antibodies, and methods for using labeled antibodies are well known in the art (see, for example, Harlow and Lane, eds. (Antibodies: A Laboratory Manual (1988) Cold Spring Harbor Laboratory Press, Cold Spring Harbor, N.Y.)).

Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions or classes, e.g., gamma (of about 330 amino acids). The subunit structures and three-dimensional configurations of different classes of immunoglobulins are well known.

In another embodiment, the terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), a toxin, e.g. tetanus toxoid, and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. All of this is well know in the arts. The antibody elicited by the methods provided herein can include whole antibodies, antibody fragments, or subfragments. In one embodiment, the antibody elicited by the methods provided herein is an immunoglobulin G.

In one embodiment, the invention provides a method for using an S. pneumoniae antigen to produce polyclonal antibodies or monoclonal antibodies (mouse or human) that cross-react with Staphylococcus strains to inhibit, suppress, prevent, or treat an S. aureus colonization and/or infection. Protocols for producing these antibodies are described in Ausubel, et al. (eds.), Molecular Cloning: A Laboratory Manual, Cold Spring Harbor Laboratory, (Cold Spring Harbor, N.Y.)., Chapter 11; in METHODS OF HYBRIDOMA FORMATION 257-271, Bartal & Hirshaut (eds.), Humana Press, Clifton, N.J. (1988); in Vitetta et al., Immunol. Rev. 62:159-83 (1982); and in Raso, Immunol. Rev. 62:93-117 (1982).

The antibodies induced in this fashion can be harvested and isolated to the extent desired by well known techniques, such as by alcohol fractionation and column chromatography, or by immunoaffinity chromatography; that is, by binding antigen to a chromatographic column packing like Sephadex™, passing the antiserum through the column, thereby retaining specific antibodies and separating out other immunoglobulins (IgGs) and contaminants, and then recovering purified antibodies by elution with a chaotropic agent, optionally followed by further purification, for example, by passage through a column of bound blood group antigens or other non-pathogen species. This procedure may be preferred when isolating the desired antibodies from the sera or plasma of humans that have developed an antibody titer against the pathogen in question, thus assuring the retention of antibodies that are capable of binding to the antigen. They can then be used in preparations for passive immunization against strains of Staphylococcus that carry the target protein to which the antibodies cross-react with.

Typically, inoculum for polyclonal antibody production typically is prepared by dispersing the antigen-immunocarrier in a physiologically-tolerable diluent such as saline, to form an aqueous composition. An immunostimulatory amount of inoculum, with or without adjuvant, is administered to a mammal and the inoculated mammal is then maintained for a time period sufficient for the antigen to induce protecting anti-antigen antibodies. Boosting doses of the antigen-immunocarrier may be used in individuals that are not already primed to respond to the antigen.

Antibodies can include antibody preparations from a variety of commonly used animals, e.g., goats, primates, donkeys, swine, rabbits, horses, hens, guinea pigs, rats, and mice, and even human antibodies after appropriate selection, fractionation and purification. Animal antisera may also be raised by inoculating the animals with formalin-killed strains of Staphylococcus that carry the antigen, by conventional methods, bleeding the animals and recovering serum or plasma for further processing.

The vaccine compositions produced according to the present description can be used by immunization to induce an immune response for the prevention or treatment of colonization and/or infection by strains of Staphylococcus that cross-react with the antibodies. In this regard, the antibody preparation can be a polyclonal composition. Such a polyclonal composition includes antibodies that bind to the antigen, and additionally may include antibodies that bind to the antigens that characterize other strains of Staphylococcus. The polyclonal antibody component can be a polyclonal antiserum, preferably affinity purified, from an animal which has been challenged with the antigen, and possibly also with other Staphylococcus targets.

In one embodiment, the Streptococcus of the invention includes but is not limited to: S. pyogenes, S. mutans, S. agalactiae, S. viridans, S. salivarus, S. thermophilus, S. mitis, or S. lactis or any other known Streptococcal species. In another embodiment, the preferred Streptococcal strain is S. pneumoniae.

In another embodiment, the Staphylococcus strain of the invention is any infectious strain known in the art, preferably a methicillin-susceptible (MSSA) or a methicillin-resistant (MRSA) strain. The bacteria of the genus Staphylococcus can, in particular, be Staphylococcus arlettae; Staphylococcus auricularis; Staphylococcus capitis.capitis; Staphylococcus capitis.ureolyticus; Staphylococcus caprae; Staphylococcus carnosus carnosus; Staphylococcus carnosus utilis; Staphylococcus chromogenes; Staphylococcus cohnii cohnii; Staphylococcus cohnii urealyticum; Staphylococcus condimenti; Staphylococcus delphini; Staphylococcus epidermidis; Staphylococcus equorum; Staphylococcus gallinarum; Staphylococcus haemolyticus; Staphylococcus hominis.hominis; Staphylococcus hominis.novobiosepticus; Staphylococcus hyicus; Staphylococcus intermedius; Staphylococcus kloosii; Staphylococcus lentus; Staphylococcus lugdunensis; Staphylococcus pasteuri; Staphylococcus piscifermentans; Staphylococcus pulvereri; Staphylococcus saprophyticus.bovis; Staphylococcus saprophyticus.saprophyticus; Staphylococcus schleiferi.coagulans; Staphylococcus schleiferi.schleiferi; Staphylococcus sciuri; Staphylococcus simulans; Staphylococcus vitulinus; Staphylococcus warneri and Staphylococcus xylosus bacteria. In a preferred embodiment the Staphylococcus strain of the invention is Staphylococcus aureus.

In another embodiment, the antibody isolated from said antibody response is an immunoglobulin. In one embodiment, the term “isolated” refers to a compound of interest (e.g., either a polynucleotide or a polypeptide) that is in an environment different from that in which the compound naturally occurs. “Isolated” is meant to include compounds that are within samples that are substantially enriched for the compound of interest and/or in which the compound of interest is partially or substantially purified.

In one embodiment, the term “cross-reaction” refers to the ability of an antibody to react with or bind to an antigen that did not stimulate its production. This can be determined using methods very well know in the art, such as western blots, where an antibody detects other proteins other than the one the antibody is specific for.

In one embodiment, the humoral response by the methods and compositions provided herein effects a cross-reactive antibody response against an S. aureus target protein. In one embodiment, the antibody response generates a cross-reactive antibody response against an S. aureus target protein In another embodiment a “cross-reactive” antibody is an antibody that reacts with an antigen other than the one that induced its production. In another embodiment, the antigen is SP_{—}1119. In another embodiment, the S. aureus target protein is a surface exposed protein that includes, but is not limited to, 1-pyrroline-5-carboxylate dehydrogenase (P5CDH) or dihydrolipoamide dehydrogenase (DLDH). Thus other surface exposed proteins on S. aureus that cross-react with S. pneumoniae-elicited antibodies are encompassed in the invention.

In one embodiment, the pneumococcal proteins with epitopes homologous to the S. aureus protein target are provided.

In one embodiment, the S. aureus protein target P5CDH is homologous to the S. pneumoniae antigen SP_{—}1119.

In another embodiment, the term “homology,” when in reference to any nucleic acid or amino acid sequence similarly indicates a percentage of nucleotides or amino acids in a candidate sequence that are identical with the nucleotides or amino acids of a corresponding native nucleic acid or amino acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from of greater than 70%. In another embodiment, “homology” refers to identity to a sequence selected from of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7. 6), 5×Denhardt's solution, 10% dextran sulfate, and 20 μg/ml denatured, sheared salmon sperm DNA.

The S. pneumoniae or S. aureus proteins of the present invention may therefore be more conveniently prepared by means of recombinant biotechnological means, whereby the gene for the S. pneumoniae or S. aureus protein of interest is isolated and inserted into an appropriate expression vector system (such as a plasmid or phage), which is then introduced into a host cell that will permit large-scale production of said protein by means of, for example, overexpression. It is to be understood that these proteins may be used to elicit antibodies that cross react with S. aureus and that can be useful for treating an S. aureus colonization and/or infection.

As a first stage, the location of the genes of interest within the S. pneumoniae genome may be determined by reference to a complete-genome database such as the TIGR database that is maintained by the Institute for Genomic Research. The selected sequence may, where appropriate, be isolated directly by the use of appropriate restriction endonucleases, or more effectively by means of PCR amplification. Suitable techniques are described in, for example, U.S. Pat. Nos. 4,683,195, 4,683,202, 4,800,159, 4,965,188, as well as in Innis et al. eds., PCR Protocols: A guide to method and applications.

Following amplification and/or restriction endonuclease digestion, the desired gene or gene fragment is ligated either initially into a cloning vector, or directly into an expression vector that is appropriate for the chosen host cell type. In the case of the S. pneumoniae proteins, Escherichia coli is the most useful expression host. However, many other cell types may be also be usefully employed including other bacteria, yeast cells, insect cells and mammalian cell systems.

High-level expression of the desired protein within the host cell may be achieved in several different ways (depending on the chosen expression vector) including expression as a fusion protein (e.g. with factor Xa or thrombin), expression as a His-tagged protein, dual vector systems, expression systems leading to incorporation of the recombinant protein inside inclusion bodies etc. The recombinant protein will then need to be isolated from the cell membrane, interior, inclusion body or (in the case of secreted proteins) the culture medium, by one of the many methods known in the art. All of the above recombinant DNA and protein purification techniques are well known to all skilled artisans in the field, the details of said techniques being described in many standard works including “Molecular cloning: a laboratory manual” by Sambrook, J., Fritsch, E. F. & Maniatis, T., Cold Spring Harbor, N.Y., 2.sup.nd ed., 1989, which is incorporated herein by reference in its entirety.

In an alternative embodiment, cells, such as the S. pneumoniae provided herein, that carry the antigen are used in a whole cell vaccine. Cells that carry the antigen can be identified and selected for use in the whole cell vaccine by using antibodies to the strain known to carry the antigen, for e.g. by using an antibody for the isolated antigen. In another embodiment, the antibody is a polyclonal antibody previously isolated from an immune response elicited by the same cell that is to be identified. In another embodiment, the antibody is a monoclonal antibody specific for the antigen. In this regard, a simple slide agglutination experiment in which antibodies to the antigen are mixed with cells can be used.

For the purified antigen vaccine above, the whole cell vaccine also comprises a pharmaceutically acceptable carrier. The whole cell vaccine also optionally may contain conventional vaccine additives like diluents, adjuvants, antioxidants, preservatives and solubilizing agents. In another embodiment, the whole cell vaccine contains only cells which carry the antigen, and does not include cells from strains of Staphylococcus that do not carry the antigen.

An advantage of the invention is that, a first S. pneumoniae antibody immune response can unexpectedly provide cross-protection against colonization and/or infection with an S. aureus strain. Such cross-protection can increase the effectiveness in treating or preventing colonization and/or infection by more than one S. aureus strain. In another embodiment, the strains encompassed within the invention include but are not limited to the following strains: 8325-4, 8325-4 Δspa, SH1000, Newman, Reynolds, Becker, USA300, MW2, and COL (see Example 3).

The term “treatment” or “treating” means any therapeutic intervention in a mammal, preferably a human, including: (i) prevention, that is, causing the clinical symptoms not to develop, e.g., preventing infection or colonization from occurring and/or developing to a harmful state; (ii) inhibition, that is, arresting the development of clinical symptoms, e.g., stopping an ongoing infection so that the infection is eliminated completely or to the degree that it is no longer harmful; and/or (iii) relief, that is, causing the regression of clinical symptoms, e.g., causing a relief of fever and/or inflammation caused by an infection.

Treatment is generally applied to any mammal susceptible to of having an S. aureus colonization and/or infection (e.g., mammals, birds, etc.), generally a mammal, usually a human where the treatment can be applied for prevention of bacterial colonization and/or infection of for amelioration of active bacterial colonization and/or infection, where the bacteria is a Staphylococcus bacteria, specifically Staphylococcus aureus.

In one embodiment, the terms “effective amount” and/or “therapeutic amount” refer to a dosage sufficient to provide treatment for the disease state being treated. This will vary depending on the patient, the disease and the treatment being effected. In the case of a bacterial colonization and/or infection, an “effective amount” is that amount necessary to substantially improve the likelihood of treating the colonization and/or infection, in particular that amount which improves the likelihood of successfully preventing colonization and/or infection or eliminating infection when it has occurred.

The term “protein” is intended to encompass any amino acid sequence and include modified sequences (e.g., glycosylated, PEGylated, containing conservative amino acid substitutions, etc.). The term includes naturally occurring (e.g., non-recombinant) proteins polypeptides, peptides, (particularly those isolated from a Staphylococcus bacteria, more particularly from Staphylococcus aureus), and oligopeptides, as well as those which are recombinantly or synthetically synthesized according to methods well known in the art. Further, the term is intended to cover naturally occurring proteins which occur in Streptococcus or Staphylococcus species and useful in treating colonization and/or infection or in generating antibodies useful in treating colonization and/or infection. Where “polypeptide” or “protein” are recited herein to refer to an amino acid sequence of a naturally-occurring protein molecule, “polypeptide,” “protein,” and like terms are not meant to limit the amino acid sequence to the complete, native amino acid sequence associated with the recited protein molecule. In addition, the polypeptides and proteins of the invention, or fragments thereof, can be generated in synthetic form having D-amino acids rather than the naturally occurring L-amino acids.

An immunogenic amount of the vaccine provided herein is typically administered to hosts having or at risk of having a staphylococcal infection such as an S. aureus infection. The hosts are typically human patients. Animals may also be treated with the compositions of the invention, including but not limited to animals of commercial or veterinary importance such as cows, sheep, and pigs, and experimental animals such as rats, mice, or guinea pigs.

An “immunogenic amount” is an amount of the S. pneumoniae sufficient to evoke an immune response that is cross-reactive to in a prophylactic or therapeutic manner against a S. aureus colonization and/or infection in the subject to which the vaccine is administered. An amount of about 10² to 10⁷ per dose is suitable, more or less can be used depending upon the age and species of the subject being treated.

Various embodiments of dosage ranges are contemplated by this invention. In one embodiment, in the case of vaccine composition, the dosage is in the range of 0.4 LD₅₀/dose. In another embodiment, the dosage is from about 0.4-4.9 LD₅₀/dose. In another embodiment the dosage is from about 0.5-0.59 LD₅₀/dose. In another embodiment the dosage is from about 0.6-0.69 LD₅₀/dose. In another embodiment the dosage is from about 0.7-0.79 LD₅₀/dose. In another embodiment the dosage is about 0.8 LD₅₀/dose. In another embodiment, the dosage is 0.4 LD₅₀/dose to 0.8 of the LD₅₀/dose.

In another embodiment, the dosage is 10⁷ bacteria/dose. In another embodiment, the dosage is 1.5×10⁷ bacteria/dose. In another embodiment, the dosage is 2×10⁷ bacteria/dose. In another embodiment, the dosage is 3×10⁷ bacteria/dose. In another embodiment, the dosage is 4×10⁷ bacteria/dose. In another embodiment, the dosage is 6×10⁷ bacteria/dose. In another embodiment, the dosage is 8×10⁷ bacteria/dose. In another embodiment, the dosage is 1×10⁸ bacteria/dose. In another embodiment, the dosage is 1.5×10⁸ bacteria/dose. In another embodiment, the dosage is 2×10⁸ bacteria/dose. In another embodiment, the dosage is 3×10⁸ bacteria/dose. In another embodiment, the dosage is 4×10⁸ bacteria/dose. In another embodiment, the dosage is 6×10⁸ bacteria/dose. In another embodiment, the dosage is 8×10⁸ bacteria/dose. In another embodiment, the dosage is 1×10⁹ bacteria/dose. In another embodiment, the dosage is 1.5×10⁹ bacteria/dose. In another embodiment, the dosage is 2×10⁹ bacteria/dose. In another embodiment, the dosage is 3×10⁹ bacteria/dose. In another embodiment, the dosage is 5×10⁹ bacteria/dose. In another embodiment, the dosage is 6×10⁹ bacteria/dose. In another embodiment, the dosage is 8×10⁹ bacteria/dose. In another embodiment, the dosage is 1×10¹⁰ bacteria/dose. In another embodiment, the dosage is 1.5×10¹⁰ bacteria/dose. In another embodiment, the dosage is 2×10¹⁰ bacteria/dose. In another embodiment, the dosage is 3×10¹⁰ bacteria/dose. In another embodiment, the dosage is 5×10¹⁰ bacteria/dose. In another embodiment, the dosage is 6×10¹⁰ bacteria/dose. In another embodiment, the dosage is 8×10¹⁰ bacteria/dose. In another embodiment, the dosage is 8×10⁹ bacteria/dose. In another embodiment, the dosage is 1×10¹¹ bacteria/dose. In another embodiment, the dosage is 1.5×10¹¹ bacteria/dose. In another embodiment, the dosage is 2×10¹¹ bacteria/dose. In another embodiment, the dosage is 3×10¹¹ bacteria/dose. In another embodiment, the dosage is 5×10¹¹ bacteria/dose. In another embodiment, the dosage is 6×10¹¹ bacteria/dose. In another embodiment, the dosage is 8×10¹¹ bacteria/dose. Each possibility represents a separate embodiment of the present invention.

By another approach, a vaccine of the present invention can be administered to a subject who then acts as a source for globulin, produced in response to challenge from the specific vaccine (“hyperimmune globulin”), that contains antibodies directed against Staphylococcus. A subject thus treated would donate plasma from which hyperimmune globulin would then be obtained, via conventional plasma-fractionation methodology, and administered to another subject in order to impart resistance against or to treat Staphylococcus colonization and/or infection. Hyperimmune globulins according to the invention are particularly useful for immune-compromised individuals, for individuals undergoing invasive procedures or where time does not permit the individual to produce his own antibodies in response to vaccination.

In another embodiment, the vaccination method provided herein is safe to use in both, immunocompetent and immunocompromised individuals.

Typically, the compositions of the invention are administered on a daily basis for at least a period of 15 days. In one embodiment, “therapeutic dose” is a dose which prevents, alleviates, abates, or otherwise reduces the severity of symptoms in a patient. The compositions of the invention may be used prophylactically to prevent staphylococcal colonization and/or infections or may be therapeutically used after the onset of symptoms. In some embodiments, induction of the formation of antibodies to the administered compound is desirable. In such instances, standard immunization protocols used in the art are preferred. The compositions administered for immunization may optionally include adjuvants.

The compositions of the invention may be administered in a variety of unit dosage forms depending on the method of administration. For example, unit dosage forms suitable for oral administration include solid dosage forms such as powder, tablets, pills, and capsules, and liquid dosage forms, such as elixirs, syrups, and suspensions. The active ingredients may also be administered parenterally in sterile liquid dosage forms. Gelatin capsules contain the active ingredient and as inactive ingredients powdered carriers, such as glucose, lactose, sucrose, mannitol, starch, cellulose or cellulose derivatives, magnesium stearate, stearic acid, sodium saccharin, talcum, magnesium carbonate and the like.

Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The concentration of the compositions of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more compositions of the invention of the invention, and more preferably at a concentration of 25% 75%.

For aerosol administration, the compositions of the invention are supplied in finely divided form along with a surfactant and propellant. Typical percentages of compositions of the invention are 0.01% 20% by weight, preferably 1% 10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides maybe employed. The surfactant may constitute 0.1% 20% by weight of the composition, preferably 0.25-5%. The balance of the composition is ordinarily propellant. A carrier can also be included, as desired, as with, e.g., lecithin for intranasal delivery.

The constructs of the invention can additionally be delivered in a depot-type system, an encapsulated form, or an implant by techniques well-known in the art. Similarly, the constructs can be delivered via a pump to a tissue of interest.

Any of the foregoing vaccine formulations may be appropriate in treatments and therapies in accordance with the present invention, provided that the active agent in the formulation is not inactivated by the formulation and the formulation is physiologically compatible.

Vaccine compositions may further incorporate additional substances to stabilize pH, or to function as adjuvants, wetting agents, or emulsifying agents, which can serve to improve the effectiveness of the vaccine.

For production of polyclonal antibodies, an appropriate target immune system is selected, typically a mouse or rabbit, although other species such as goats, sheep, cows, guinea pigs, and rats maybe used. The substantially purified antigen is presented to the immune system according to methods known in the art. The immunological response is typically assayed by an immunoassay. Suitable examples include ELISA, RIA, fluorescent assay, or the like. These antibodies will find use in diagnostic assays or as an active ingredient in a pharmaceutical composition.

The vaccine of the invention can be parentarally administrated. Parenteral administration is generally characterized by injection, either subcutaneously, intradermally, intramuscularly, or intravenously, preferably subcutaneously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as for example, sodium acetate, sorbitan monolaurate, triethanolamine oleate, cyclodextrins, and the like.

The percentage of active ingredient contained in such parental compositions is highly dependent on the specific nature thereof, as well as the needs of the subject. However, percentages of active ingredient of 0.01% to 10% in solution are employable, and will be higher if the composition is a solid which will be subsequently diluted to the above percentages. Preferably, the composition will comprise 0.2-2% of the active ingredient in solution.

Another approach for parental administration employs the implantation of a slow-release or sustained-release system, such that a constant level of dosage is maintained. Various matrices (e.g., polymers, hydrophilic gels, and the like) for controlling the sustained release, and for progressively diminishing the rate of release of active agents are known in the art. See U.S. Pat. No. 3,845,770 (describing elementary osmotic pumps); U.S. Pat. Nos. 3,995,651, 4,034,756 and 4,111,202 (describing miniature osmotic pumps); U.S. Pat. Nos. 4,320,759 and 4,449,983 (describing multichamber osmotic systems referred to as push-pull and push-melt osmotic pumps); and U.S. Pat. No. 5,023,088 (describing osmotic pumps patterned for the sequentially timed dispensing of various dosage units).

The vaccine provided herein may also be administered to the respiratory tract as a nasal or pulmonary inhalation aerosol or solution for a nebulizer, or as a microfine powder for inhalation, alone or in combination with an inert carrier such as lactose, or with other pharmaceutically acceptable excipients.

Vaccines according to the invention can be administered to a subject not already infected with Staphylococcus, thereby to induce a Staphylococcus-protective immune response (humoral or adaptive) in that subject. Alternatively, vaccines within the present invention can be administered to a subject in which Staphylococcus infection already has occurred but is at a sufficiently early stage that the immune response produced to the vaccine effectively inhibits further spread of infection.

The vaccine provided herein can be administered in a live form, a heat-killed form or in any form know in the art to elicit an immune response.

The amount of the vaccine administered is an amount sufficient to elicit a protective immune response in the host and can be empirically determined, as it is to be understood by a skilled artisan. Methods for determining such appropriate amounts are routine and well known in the art. The amounts effective in such animal models can be extrapolated to other hosts (e.g., livestock, humans, etc.) in order to provide for an amount effective for vaccination.

The vaccine may be given in a single dose schedule, or preferably a multiple dose schedule in which a primary course of vaccination may be with 1-10 separate doses, followed by other doses given at subsequent time intervals required to maintain and or reinforce the immune response, for example, at 1-4 months for a second dose, and if needed, a subsequent dose(s) after several months. Examples of suitable immunization schedules include: (i) 0, 1 months and 6 months, (ii) 0, 7 days and 1 month, (iii) 0 and 1 month, (iv) 0 and 6 months, or other schedules sufficient to elicit the desired immune responses expected to confer protective immunity, or reduce disease symptoms, or reduce severity of disease.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the embodiments of the invention. They should in no way be construed, however, as limiting the broad scope of the invention.

### EXAMPLES

### Materials and Methods

### Mouse Model of S. Pneumoniae Nasopharyngeal Colonization

To investigate the humoral immune response elicited by S. pneumoniae carriage, mouse model of nasopharyngeal colonization was employed. This murine model shares similar colonization dynamics and immune responses with those observed in humans. Briefly, adult mice were intranasally inoculated without anesthesia to avoid aspiration with 10⁷ CFU of PBS-washed, mid-log S. pneumoniae by atraumatic application to the nares. Colonization was allowed to clear over the course of five weeks, at which point the animals were sacrificed for the collection of sera and nasal lavage fluid. To minimize the confounding effect of variation in antibody profiles between animals, pre-colonization sera were obtained by tail bleed prior to S. pneumoniae inoculation for comparison with sera gathered post-colonization.

### Bacteria and Growth Conditions

Unless otherwise specified, all S. aureus strains were grown to mid-log phase (OD₆₂₀=0.4) in BHI media at 37° C. with shaking. All S. pneumoniae strains were grown to mid-log phase (OD₆₂₀=0.4) in TS media at 37° C. without shaking. The genome sequences of all strains used in this study are publicly available.

### Protein Gel Electrophoresis and Staining

One dimensional sodium dodecyl sulphate polyacrylamide gel electrophoresis (SDS-PAGE) was performed using the Mini-Protean II system (BioRad). Protein samples were suspended in Laemmli sample buffer and boiled for 5 min prior to electrophoresis at 100 V. Two dimensional SDS-PAGE involved separation of proteins by isoelectric point and by molecular weight, respectively. Isoelectric focusing (pI 4.7-5.9) was carried out in a Protean IEF cell (BioRad), using 7 mm ReadyStrips, according to the manufacturer's instructions. The proteins were then separated in the second dimension using 10% polyacrylamide gels in a Mini-Protean II system. The proteins were stained in the gels using Coomassie brilliant blue R-250 (Fisher Scientific, Pittsburgh, Pa.).

### Western Blot Analysis

Protein mixtures were separated by one-dimensional and two-dimensional SDS-PAGE and transferred to PVDF membrane (Thermo Scientific, Waltham, Mass.) by Trans-Blot SD semi-dry transfer system (BioRad) at 18 V. Gels from two-dimensional SDS-PAGE were half transferred (18 V for 0.3 hr, compared to 0.6 hr for one-dimensional), and following transfer the remaining gel was stained using Coomassie brilliant blue R-250 to obtain a stained gel and Western blot membrane pair. Membranes were then blocked in PBS supplemented with 1% BSA prior to incubation with mouse serum at 1:500 dilution at room temperature overnight. Bound antibody was detected by anti-mouse secondary antibody conjugated to alkaline phosphatase (Sigma), and BCIP/NBT (Fisher) development.

### Mass Spectroscopy

Spots identified by Western blot as cross-reactive were traced on the corresponding Coomassie stained gel, and proteins spots of interest were excised from the gel using a pipette tip. The protein within the gel plug was trypsin digested and injected onto a HPLC C18 column to separate the digested peptides. The separated peptides were sprayed into an LTQ ion-trap mass spectrometer (Thermo Scientific). Mascot software (Mascot Software Technologies, Bloomington, Ind.) was used to search bacterial databases for sequence similarity. Cutoffs were assigned as a protein score of >70 with a unique peptide value of >2.

### Example 1

### Identification of S. Aureus Protein Targets of Cross-Reactive Pneumococcal Antibody

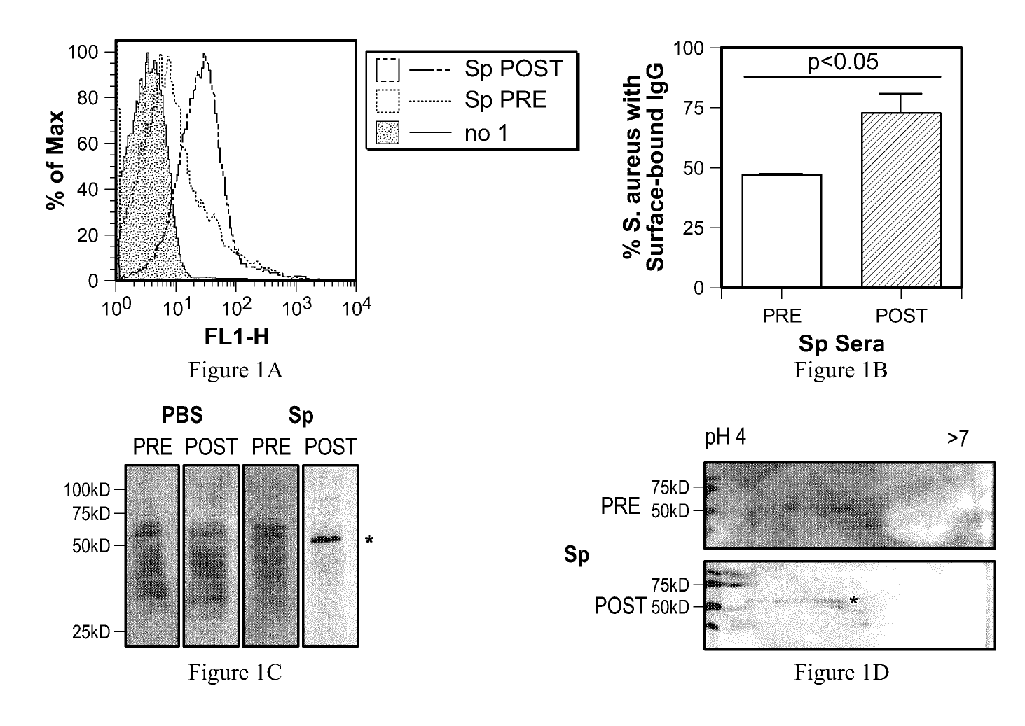

To determine whether cross-reactive antibodies recognize the surface of S. aureus, flow cytometry was used measure surface-bound IgG on live S. aureus. S. aureus 8325-4 Δspa cells were incubated with sera from mice before or after colonization with S. pneumoniae TIGR4 (FIG. 1). Following a wash to remove unbound antibody, surface-bound IgG was labeled using FITC-conjugated anti-mouse IgG and analyzed on a FACS Calibur. The percent of S. aureus cells with surface-bound IgG was calculated by comparison to a no sera control.

To identify specific S. aureus protein targets of cross-reactive pneumococcal antibody, western immunoblots were used to probe S. aureus 8325-4 Δspa lysates with a 1:500 dilution of sera taken from mice before and after intranasal colonization by S. pneumoniae TIGR4. Sera from mice inoculated with PBS were used as negative controls. For all western blots, S. aureus lysates were made by incubating live cells at 37° C. with commercially available lysostaphin. Lysates were mixed with equal volumes of laemmli sample buffer and boiled for 5 min prior to loading in 10% polyacrylamide gels. Proteins separated by SDS-PAGE were then transferred to PVDF membranes prior to incubation with antibody. Candidate S. aureus proteins were identified as bands that were novel or enhanced in western blots with pneumococcal antisera compared to pre-colonization and PBS controls. Because variation in antibody responses varies from mouse to mouse, as in humans, S. aureus targets that were common to a statistically significant majority of animals were prioritized.

To further isolate specific S. aureus targets, two-dimensional SDS-PAGE and was used then half-transferred to PVDF membranes for western blots as described above. Proteins targeted by pneumococcal antibodies were excised from the original 2D gel and sent for mass spectrometry analysis at the UPENN Proteomics core facility (Table 1).

TABLE 1
Candidate S. aureus targets identified by MS

| | 1-pyrroline-5-carboxylate | Dihydrolipoamide |
|---|---|---|
| | dehydrogenase | dehydrogenase |
| Abbreviation | P5CDH | DLDH |
| MS score | 3808 | 3322 |
| emPAI | 3.04 | 2.98 |
| Predicted MW | 56 | 50 |
| pI | 4.98 | 4.95 |
| KEGG | 1. Oxidoreductases | 1. Oxidoreductases |
| EC#/Function | 1.5 Acting on the CH-NH | 1.8 Acting on a sulfur |
| | group of donors | group of donors |
| | 1.5.1 With NAD+ or | 1.8.1 With NAD+ or |
| | NADP+ as acceptor | NADP+ as acceptor |
| | 1.5.1.12 1-pyrroline-5- | 1.8.1.4 dihydrolipoyl |
| | carboxylate | dehydrogenase |
| | dehydrogenase | |

N.B.
MS score >70 considered significant;
emPAI: empirical protein abundance index

To determine the immunogenic proteins from S. pneumoniae responsible for eliciting cross-reactive antibody, the amino acid sequences of S. aureus candidates to the proteome of S. pneumoniae using BLAST-P (http://blast.ncbi.nlm.nih.gov/Blast.cgi) was compared in order to identify pneumococcal proteins with epitopes homologous to the S. aureus protein target.

It was found that pneumococcal colonization induces antibodies that cross-react with S. aureus surface proteins (FIG. 1).

### Example 2

### Expression of Candidate Proteins and Generation of Specific Antisera

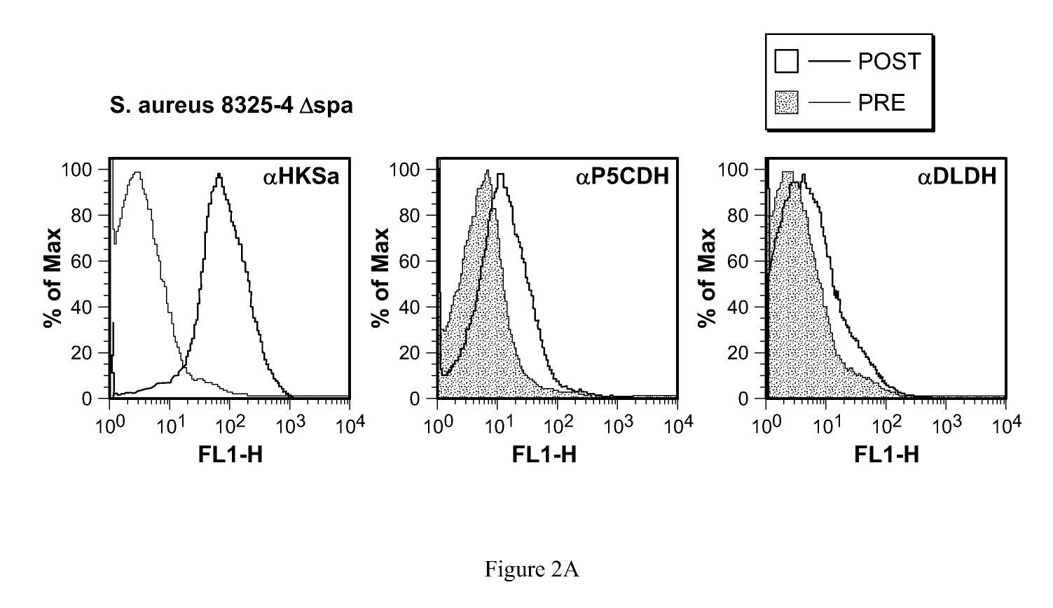

Once S. aureus target proteins P5CDH and DLDH and their S. pneumoniae homologs were identified (FIG. 2), each of proteins were expressed independently for further study. Standard pET vector (pET-29b) expression technology was used in E. coli BL21 to generate polyhistidine tagged recombinant constructs of each candidate. His-tags were used for purification of desired proteins using immobilized metal affinity chelate chromatography. Inclusion of a thrombin cleavage site between the protein of interest and its his-tag allowed for his-tag removal by thrombin following protein purification. Antisera specific to the purified protein candidates were generated at Cocalico Biologicals, Inc. by immunizing two New Zealand white rabbits with each S. aureus target or S. pneumoniae immunogen, according to standard commercial protocols.

### Example 3

### Confirmation of Accessibility and Conservation of S. Aureus Targets

To confirm the surface exposure of S. aureus candidates, specific P5CDH and DLDH antisera was incubated with live S. aureus 8325-4 Δspa for 1 hr at 37° C. Bacterial cells were then washed and incubated with AP-conjugated anti-rabbit IgG to detect surface bound IgG by flow cytometry. Naïve rabbit sera was used as a control. To determine the surface exposure of S. pneumoniae candidates, specific SP_{—}1119 and SP_{—}1161 antisera was incubated with live S. pneumoniae TIGR4WT and Δcps. Bacterial cells were then washed and incubated with AP-conjugated anti-rabbit IgG to detect surface bound IgG by flow cytometry. Naïve rabbit sera was used as a control (FIG. 2).

To determine the degree of conservation of candidate S. pneumoniae immunogens and S. aureus targets, genomic comparisons were made using the numerous publicly accessible whole genome sequences for each species (Table 2). DLDH and P5CDH antisera were used in western blots to look for the presence of specific S. aureus targets in whole cell lysates of clinical S. aureus isolates.

TABLE 2
Candidate S. pneumoniae antigens identified by sequence homology.

| | | | BLAST | | | | |
|---|---|---|---|---|---|---|---|
| S. aureus | S. pneumoniae homolog | | | E | Predicted | % | KEGG |
| target | Locus | Annotation | Score | Value | MW | identity | SW-score |
| P5CDH | SP_1119 | Putative | 213 | 1e−56 | 51.1 kD | 30%* | 749 |
| | | GAPDH | | | | | |
| DLDH | SP_1161 | Putative | 250 | 6e−68 | 60.4 kD | 37%** | 956 |
| | | DLDH | | | | | |

*members of same enzyme superfamily (aldehyde dehydrogenase)
**Functionally homologous enzymes in related multi-enzyme complexes (both in 2-oxo-acid dehydrogenase complex family)

### Example 4

### Assessing Cross-Reactivity Between S. Aureus and Pneumoniae Proteins

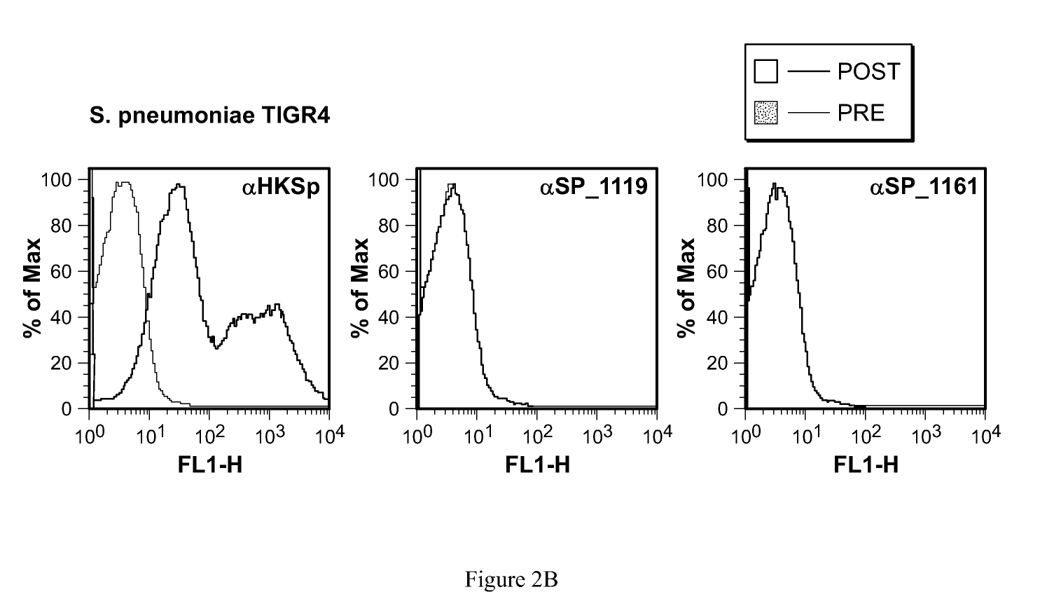

To determine if antibodies specific to S. pneumoniae proteins SP_{—}1119 and SP_{—}1161 cross-react with live S. aureus, whole S. aureus 8325-4 Δspa were incubated with specific rabbit antisera to SP_{—}1119 and SP_{—}1161, respectively. Bacterial cells were then washed and incubated with AP-conjugated anti-rabbit IgG to detect surface bound IgG by flow cytometry. Naïve rabbit sera was used as a control. The inverse experiments using live S. pneumoniae TIGR4Δcps and specific antisera to S. aureus proteins DLDH and P5CDH were performed in the same manner. Results show that antibodies raised against pneumococcal protein SP_{—}1119, but not SP_{—}1161, bind to the surface of S. aureus (FIG. 3).

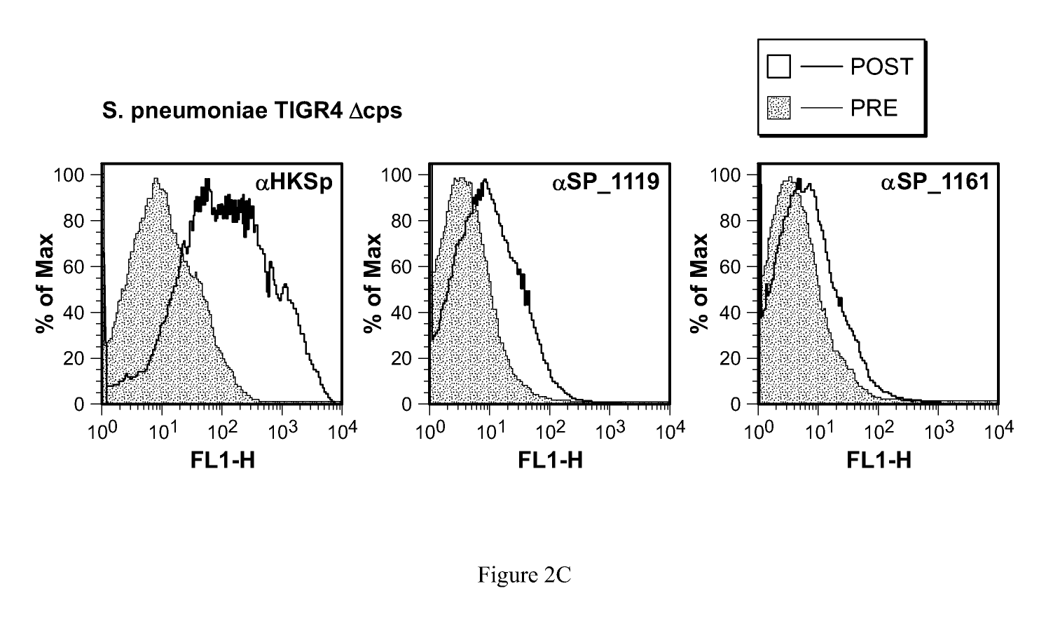

To determine whether pneumococcal colonization elicited antibodies to candidate protein antigens, sera from mice pre- and post-pneumococcal colonization and mock PBS colonization were used in western blots against purified proteins DLDH, P5CDH, SP_{—}1119 and SP_{—}1161. It was observed that pneumococcal colonization elicits antibodies that cross-react with S. aureus protein P5CDH (FIG. 4).

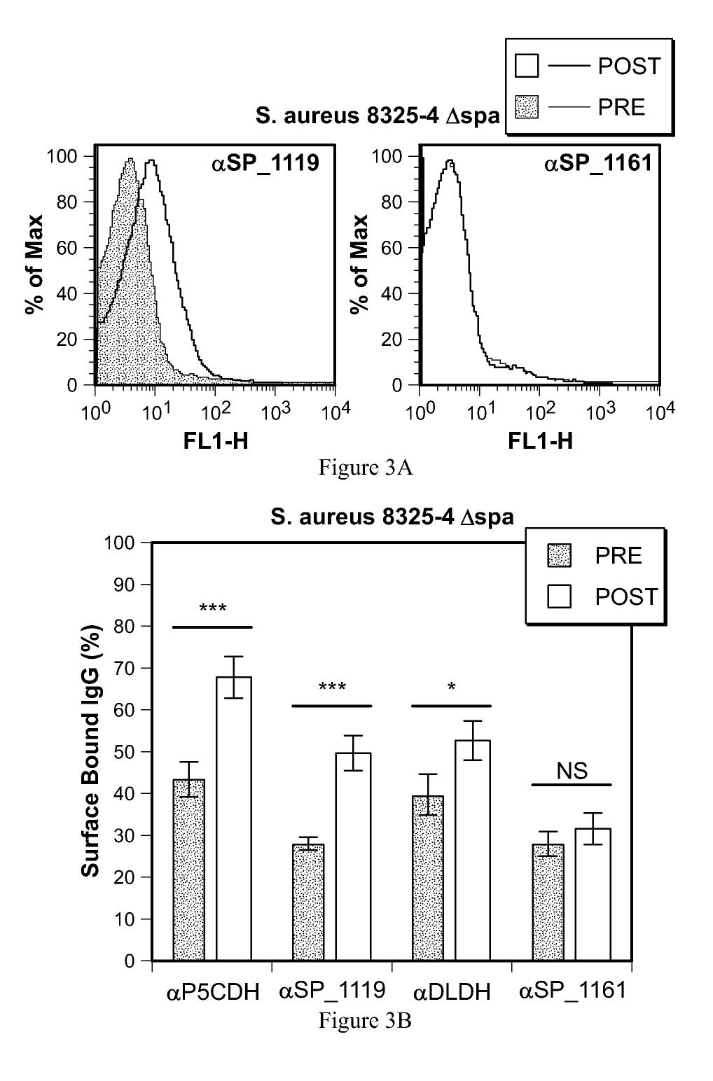

To date, the S. aureus strains tested are: 8325-4, 8325-4 Δspa, SH1000, Newman, Reynolds, Becker, USA300, MW2, and COL. Results show that S. aureus antigens DLDH and P5CDH are broadly conserved (FIG. 5).

### Example 5

### Characterizing Antibody Responses to Pneumococcal Carriage that Cross-React with Staphylococcus aureus

Staphylococcus aureus is a bacterial pathogen responsible for significant morbidity, mortality, and excess healthcare cost worldwide. Given that the treatment of S. aureus colonization and/or infection has become increasingly difficult due to the rising prevalence of methicillin-resistant S. aureus (MRSA) and the lack of a vaccine against this pathogen, there is an urgent need for novel approaches to prevent S. aureus infection and transmission. The predominant risk factor for S. aureus infection and transmission is asymptomatic colonization of the nasal mucosa. However, the specific host and bacterial determinants of S. aureus carriage are not well understood. Significantly reduced S. aureus colonization rates have recently been associated with carriage of another member of the nasopharyngeal flora, Streptococcus pneumoniae (the pneumococcus). Pneumococcal colonization in healthy children reduces the odds of nasal carriage of S. aureus by half, but has no effect in immunocompromised individuals, implicating a role for the host immune response in mediating this interference phenomenon. In humans and a murine model, asymptomatic pneumococcal colonization elicits a robust serum antibody response.

A mouse model of pneumococcal colonization was chosen that shares carriage dynamics and immune responses with human colonization. Using this model, it was first asked whether pneumococcal colonization elicits antibodies capable of recognizing the surface of S. aureus. Live S. aureus cells were incubated with sera from mice taken before (pre) and after (post) pneumococcal colonization and a significant increase (post vs. pre) in total IgG binding was observed, as determined by flow cytometry. To identify which staphylococcal protein(s) were targeted by pneumococcal antibodies, western blots were performed using S. aureus lysates that were probed with sera from mice before and after pneumococcal colonization. In a significant percentage of the mice tested, antibody binding post-pneumococcal colonization to a candidate S. aureus protein between 50 and 75 kd in size was observed.

Mass spectrometric analysis identified this band as two proteins, P5CDH and DLDH, that were indistinguishable by 2D gel electrophoresis. Both of these are dehydrogenases and have been identified as surface associated in the literature. For each staphylococcal protein, there is one pneumococcal homolog as identified by BLAST and KEGG alignments, SP_{—}1119 and SP_{—}1161 respectively, both putative dehydrogenases.

To test whether any of these proteins play a role in mediating cross-reactive antibody responses, each protein was expressed recombinantly and subsequently purified using His-tag affinity chromatography. In western blots of recombinant proteins, a boost in antibody binding post-pneumococcal colonization to the homologous pair SP_{—}1119 (Spn) and P5CDH (Sa) was observed. Specific antisera to each of the four proteins in rabbits was also generated. Using this antisera in flow cytometry of live bacterial cells, it was confirmed that both P5CDH and DLDH are surface-exposed in S. aureus and that SP_{—}1119 and SP_{—}1161 are surface-associated in S. pneumoniae but masked by capsule. When S. aureus cells were incubated with antisera to the pneumococcal proteins, cross-reactive binding with anti-SP_{—}1119 was observed but not anti-SP_{—}1161 antibodies. In the opposite direction, antibodies to the staphylococcal homolog of SP_{—}1119, P5CDH, bind to the surface of unencapsulated S. pneumoniae, while antibodies to DLDH do not. Together these data suggest that pneumococcal colonization induces antibodies that cross-react with live S. aureus, potentially due to the homology of the pneumococcal protein SP_{—}1119 with the surface-exposed staphylococcal protein P5CDH.

Next it was sought to verify that SP_{—}1119 and P5CDH contribute to the cross-reactive antibody response between S. pneumoniae and S. aureus. To confirm that the cross-reactivity that was observed was due to these proteins, a genetic approach was taken to determine whether deletion of either SP_{—}1119 or P5CDH abrogates crossreactivity by specific antisera. A double deletion mutant in S. pneumoniae that lacks both SP_{—}1119 and capsule was created and a loss in IgG binding with antisera against SP_{—}1119 and P5CDH was observed, but not SP_{—}1161, as expected. Aa P5CDH mutant in S. aureus was obtained; however, this strain cannot be used in flow cytometry due to the expression of protein A, which binds IgG non-specifically via the Fc region. Ongoing work to circumvent this confounding factor includes making F(ab′)2 fragments (that lack the Fc region) from specific antisera for use in flow cytometry with the P5CDH mutant.

To confirm quantitatively that pneumococcal colonization elicits antibodies to our proteins of interest, we performed ELISAs using sera from mice pre- and post-pneumococcal colonization and observed an increase in IgG titers to SP_{—}1119 and P5CDH post-pneumococcal colonization (as expected based on western blots described in the prior section). Moreover, there was a positive linear correlation between elevated titers to SP_{—}1119 and P5CDH. We conducted experiments to determine whether children colonized with S. pneumoniae mount serum IgG titers to SP_{—}1119 and SP_{—}1161. Studies using Luminex technology (high throughput ELISA) confirmed that both proteins are immunogenic in children colonized with S. pneumoniae at 12 and 24 months of age. Next, we wanted to assess whether cross-reactive antibodies, and anti-SP_{—}1119 and anti-P5CDH antibodies in particular, contribute to immune protection against S. aureus. Antisera against P5CDH and SP_{—}1119 had a direct effect in vitro in limiting S. aureus growth as assessed by optical density. Protection in vivo was also assessed in several animal models. In a murine model of S. aureus bacteremia, an example of systemic infection, passive immunization with P5CDH antisera protected against mortality and reduced bacterial burden in the blood. Protection was not observed following passive immunization with any other specific antisera. To assess protection against S. aureus colonization, we developed a mouse model of staphylococcal carriage using a human isolate known for its superior colonization phenotype, S. aureus 502A. In our model, we then assessed whether prior colonization with S. pneumoniae affected S. aureus carriage levels. In mice colonized with S. pneumoniae compared to PBS controls, we observed a significant decrease in S. aureus colonization levels at 7 weeks postpneumococcal colonization, a timepoint at which no pneumococci remain in the nasopharyngx. This effect was lost in mice deficient in antibody (μMT mice), implicating that cross-reactive antibody is necessary for this interference phenomenon between S. pneumoniae and S. aureus.

### Example 6

### Vaccination with SP_{—}1119 Decreases Viral Titer of S. Aureus

SP_{—}1119 and P5CDH were cloned out of the S. pneumoniae genome and expressed recombinantly in E. coli BL21 (Stratagene) using commercially available pET vector technology (Novagen). Both proteins were purified using Ni-NTA-agarose beads (Qiagen) and dialyzed into storage buffer. For intranasal immunizations with purified proteins, 4 μg of antigen was mixed with 1 μg cholera toxin as adjuvant and administered atraumatically to the nares of a C57B1/6 mouse. 10 mice were used in each immunization group. Immunization was performed 1× per week for three weeks. Two weeks after the last immunization, mice were challenged with 10⁸ CFU of S. aureus 502A intranasally. At day 1 post-challenge, mice were sacrificed and nasal lavages were taken to enumerate remaining CFU of S. aureus.

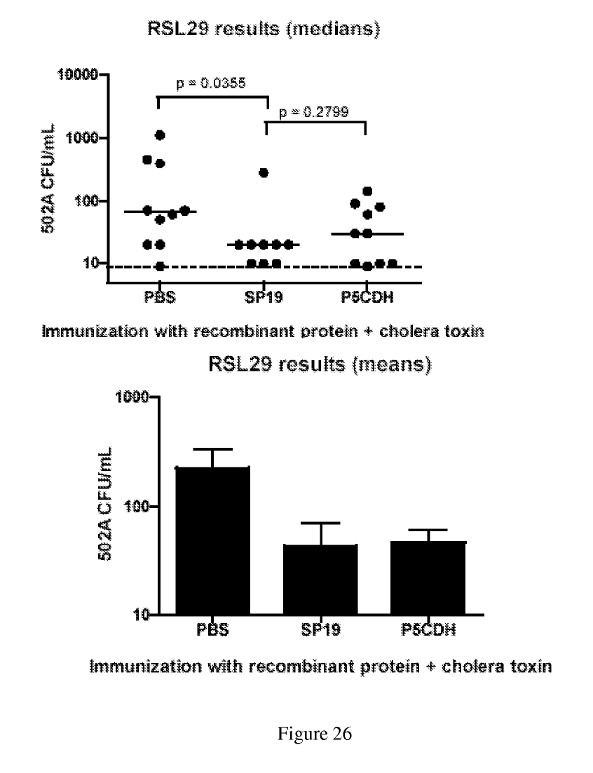

Mice that were immunized intranasally with purified protein candidate SP_{—}1119 and P5CDH were found to be significantly protected from nasal colonization (See FIG. 26). This demonstrates that SP19 is an effective vaccine that mediates in vivo protection against nasal S. aureus colonization and infection.

Having described the embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A vaccine composition comprising a recombinant protein having the amino acid sequence set forth in SEQ ID NO.: 1 or 2, wherein said composition is capable of preventing Staphylococcus aureus (S. aureus) colonization and/or infection in a subject.

2. The composition of claim 1, wherein said composition further comprises an adjuvant.

3. The composition of claim 2, wherein said ajuvant is cholera toxin.

4. A method of preventing a Staphylococcus aureus (S. aureus) colonization and/or infection in a subject, said method comprising the step of administering a therapeutically effective amount of a vaccine composition that comprises a recombinant protein having the amino acid sequence set forth in SEQ ID NO.: 1 or 2, thereby preventing said colonization and/or infection in said subject.

5. The method of claim 4, wherein said vaccine composition is administered intranasally.

6. The method of claim 4, wherein said vaccine composition further comprises an adjuvant.

7. The method of claim 6, wherein said ajuvant is cholera toxin.

8. A method of treating a disease associated with Staphylococcus aureus (S. aureus) colonization and/or infection in a subject, said method comprising the step of administering a therapeutically effective amount of a vaccine composition that comprises a recombinant protein having the amino acid sequence set forth in SEQ ID NO.: 1 or 2, thereby treating said disease in said subject.

9. The method of claim 8, wherein said vaccine composition is administered intranasally.

10. The method of claim 8, wherein said vaccine composition further comprises an adjuvant.

11. The method of claim 10, wherein said ajuvant is cholera toxin.

12. A method of eliciting an anti-S. aureus immune response in a subject, said method comprising the step of administering to said subject a therapeutically effective amount of a vaccine composition comprising a recombinant protein having the amino acid sequence set forth in SEQ ID NO.: 1 or 2, thereby eliciting said anti-S. aureus immune response in said subject.

13. The method of claim 12, wherein said vaccine composition is administered intranasally.

14. The method of claim 12, wherein said vaccine composition further comprises an adjuvant.

15. The method of claim 13, wherein said ajuvant is cholera toxin.

16. A method of preventing a Staphylococcus aureus (S. aureus) colonization and/or infection in a subject, said method comprising the step of administering a therapeutically effective amount of a first vaccine composition that comprises a recombinant protein having the amino acid sequence set forth in SEQ ID NO.: 1 and a second vaccine composition that comprises a recombinant protein having the amino acid sequence set forth in SEQ ID NO.: 2, thereby preventing said colonization and/or infection in said subject.

17. The method of claim 16, wherein said first and/or second vaccine composition is administered intranasally.

18. The method of claim 16, wherein said first and/or second vaccine composition further comprises an adjuvant.

19. The method of claim 18, wherein said ajuvant is cholera toxin.


SEQUENCE LISTING (2 sequences)

SEQ ID NO: 1 - protein, 474 aa, Streptococcus pneumoniae
MTRYQNLVNGKWKSSEQEITIYSPINQEELGTVPAMTQTEADEAMQAARAALPAWRALSAVERAAYLHKTAAILERDKEEIGTILAKEVAKGIKAAIGEVVRTADLIRYAAEEGLRITGQAMEGGGFEATSKNKLAVVRREPVGIVLAIAPFNYPVNLSASKIAPALIAGNVVMFKPPTQGSISGLLLAKAFEEAGIPAGVFNTITGRGSEIGDYIIEHKEVNFINFTGSTPIGERIGRLAGMRPIMLELGGKDAALVLEDADLEHAAKQIVAGAFSYSGQRCTAIKRVIVLESVADKLATLLQEEVSKLTVGDPFDNADITPVIDNASADFIWGLIEDAQEKEAQALTPIKREGNLLWPVLFDQVTKDMKVAWEEPFGPVLPIIRVASVEEAIAFANESEFGLQSSVFTNDFKKAFEIAEKLEVGTVHINNKTQRGPDNFPFLGVKGSGAGVQGIKYSIEAMTNVKSIVFDVK

SEQ ID NO: 2 - protein, 514 aa, Staphylococcus aureus
MVVEFKNEPGYDFSVQENVDMFKKALKDVEKELGQDIPLVINGEKIFKDDKIKSINPADTSQVIANASKATKQDVEDAFKAANEAYKSWKTWSANDRAELMLRVSAIIRRRKAEIAAIMVYEAGKPWDEAVGDAAEGIDFIEYYARSMMDLAQGKPVLDREGEHNKYFYKSIGTGVTIPPWNFPFAIMAGTTLAPVVAGNTVLLKPAEDTPYIAYKLMEILEEAGLPKGVVNFVPGDPKEIGDYLVDHKDTHFVTFTGSRATGTRIYERSAVVQEGQNFLKRVIAEMGGKDAIVVDENIDTDMAAEAIVTSAFGFSGQKCSACSRAIVHKDVYDEVLEKSIKLTKELTLGNTVDNTYMGPVINKKQFDKIKNYIEIGKEEGKLEQGGGTDDSKGYFVEPTIISGLKSKDRIMQEEIFGPVVGFVKVNDFDEAIEVANDTDYGLTGAVITNNREHWIKAVNEFDVGNLYLNRGCTSAVVGYHPFGGFKMSGTDAKTGSPDYLLHFLEQKVVSEMF